(19) United States
(12) Patent Application Publication
(10) Pub. No.: US 2022/0220566 A1
(43) Pub. Date: Jul. 14, 2022

(54) EXTRACHROMOSOMAL DNA IDENTIFICATION AND METHODS OF USE

(71) Applicant: The Jackson Laboratory, Bar Harbor, ME (US)

(72) Inventors: Chia-Lin WEI, Farmington, CT (US); Chee Hong WONG, Farmington, CT (US); Harianto TJONG, Farmington, CT (US); Roel VERHAAK, Farmington, CT (US)

(73) Assignee: The Jackson Laboratory, Bar Harbor, ME (US)

(21) Appl. No.: 17/607,635

(22) PCT Filed: Apr. 29, 2020
(86) PCT No.: PCT/US2020/030408

§ 371 Date: Oct. 29, 2021

Related U.S. Application Data
(60) Provisional application No. 62/840,735, filed on Apr. 30, 2019.

(51) Int. Cl. C12Q 1/6886; C12Q 1/6841

(52) U.S. Cl. CPC C12Q 1/6886; C12Q 2600/158; C12Q 2600/156; C12Q 1/6841

(57) ABSTRACT

The invention, in part, encompasses methods to identify extrachromosomal circular DNA (ecDNA) and methods to identify and assess interactions between ecDNA and oncogene transcription.

### RELATED APPLICATIONS

This application claims benefit under 35 U.S.C. § 119(e) of U.S. Provisional Application No. 62/840,735, filed Apr. 30, 2019, the entire contents of which is incorporated by reference herein in its entirety.

### GOVERNMENT INTEREST

This invention was made with government support under the NCI P30CA034196, U54 DK107967, UM1 HG009409, and R01 CA190121 grants awarded by the National Institutes of Health (NHI). The government has certain rights in the invention.

### FIELD OF THE INVENTION

The invention, in some aspects, relates to method of identifying extrachromosomal circular DNA (ecDNA) and methods of using ecDNA to assess transcription of target genes.

### BACKGROUND OF THE INVENTION

Extrachromosomal DNAs (ecDNAs) are extrachromosomal circular chromatin elements [Cox, D. et al. (1965) Lancet 1, 55-58; Spriggs, A. I. et al. (1962) Br Med J 2, 1431-1435]. First described as ‘double-minute chromatin bodies’ in karyotypes of cells by microscopic imaging [Cox, D. et al. (1965) Lancet 1, 55-58], ecDNAs were thought to be a mode of gene amplification associated with in vitro drug resistance [Spriggs, A. I. et al. (1962) Br Med J 2, 1431-1435; deCarvalho, A. C. et al. (2018) Nat Genet 50, 708-717; Nathanson, D. A. et al. (2014) Science 343, 72-76]. More recently, ecDNAs were found to be common in primary cancers [Xu, K. et al. (2018) Acta Neuropathol, doi:10.1007/s00401-018-1912-1], and to constitute a bona fide mechanism and adaptive reservoir for oncogene amplification [Turner, K. M. et al. (2017) Nature 543, 122-125]. EcDNA may be derived through chromothriptic genome shattering events and consequently, can consist of hundreds of DNA segments [Ma, K. et al. (2012) Int J Mol Sci 13, 11974-11999]. Their accumulation in cancer cells offers a competitive advantage in response to selective pressures in the tumor microenvironment and in response to cytotoxic therapeutic agents [Spriggs, A. I. et al. (1962) Br Med J 2, 1431-1435; Kohl, N. E. et al. (1983) Cell 35, 359-367]. Rapid fluctuation in ecDNA levels as a result of disjointed inheritance patterns [Spriggs, A. I. et al. (1962) Br Med J 2, 1431-1435] likely contributes to the mechanism of tumor evolution. Glioblastoma (GBM) is an aggressive brain tumor in which ecDNAs are frequently observed [Rausch, T. et al. (2012) Cell 148, 59-71; Xue, Y. et al. (2017) Nat Med 23, 929-937]. Previous analysis of a set of unique GBM-derived neurosphere cultures using standard genomic approaches detected multiple ecDNAs harboring oncogenes including EGFR, MYC and CDK4 [Spriggs, A. I. et al. (1962) Br Med J 2, 1431-1435]. While the presence of ecDNA and their structure information have begun to be characterized by these standard approaches, the mechanism(s) by which ecDNA are deployed to modulate cancer progression and to contribute to cancer drug resistance is not yet understood.

### SUMMARY OF THE INVENTION

According to an aspect of the invention, methods of identifying an extrachromosomal DNA (ecDNA) in a cell are provided, the methods including: (a) detecting a chromatin interaction between a non-linear DNA molecule and at least one linear chromosome of a chromosome pair, wherein the interaction comprises a contact between the non-linear DNA molecule and the at least one linear chromosome of the chromosome pair, and wherein the presence of: (i) a significantly high frequency of the detected chromatin interaction in the cell; (ii) contact between the non-linear DNA molecule and at least one linear chromosome of each of the chromosome pairs in the cell; and (iii) an increase in an average per-cell number of copies of the non-linear DNA molecule in a plurality of the cells over time, identifies the non-linear DNA molecule as an ecDNA. In some embodiments, the method also includes determining a frequency of the detected chromatin interaction. In certain embodiments, the method also includes determining size of the non-linear DNA. In some embodiments, the method also includes determining a number of copies of the non-linear DNA molecule in the cell. In some embodiments, the method also includes determining an average per-cell number of copies of the non-linear DNA molecule in a plurality of the cells at a first time and comparing the determined average to a control average per-cell number of copies of the non-linear DNA. In certain embodiments, the control average per-cell number of copies of the non-linear DNA molecule is an average per-cell number of copies of the non-linear DNA molecule determined in the plurality of cells at a different time point. In some embodiments, the method also includes determining the sequence of at least a portion of the non-linear DNA. In certain embodiments, the method also includes identifying the presence an oncogene sequence in the determined sequence. In some embodiments, the cell is a cancer cell. In some embodiments, the cell is a precancerous cell. In some embodiments, the cell is obtained from a plurality of cells comprising the cancer cells. In certain embodiments, the cell is obtained from a subject. In some embodiments, the subject is at least one of: diagnosed with, suspected of having, and at risk of having a cancer. In some embodiments, the cell is obtained from a cell culture. In certain embodiments, the cell is a vertebrate cell. In some embodiments, the cell is a mammalian cell, and optionally is a human cell.

According to another aspect of the invention, methods of identifying an ecDNA-modulated oncogene are provided, the methods including: (a) detecting an interaction between an ecDNA and one or more target genes located in a cell, wherein the detecting comprises directly measuring chromatin interactions between the ecDNA and a regulatory element of the one or more target genes; (b) identifying one or more of the target genes of the interaction detected in step (a) in which the target gene's transcription is modulated by the detected interaction; and (c) determining whether one or more of the target genes identified in step (b) is an oncogene, wherein the determination of the target gene as an oncogene identifies the oncogene as an ecDNA-modulated oncogene. In some embodiments, the identifying in step (b) includes measuring a level of transcription of the identified target gene and comparing the measured level to a control level of transcription of the target gene. In certain embodiments, the transcription modulation is an increase in transcription. In some embodiments, a means of detecting in step (a) includes a ChIA-PET method. In some embodiments, a means of detecting in step (a) includes a Hi-C method. In some embodiments, the regulatory element includes a promoter for the target gene. In certain embodiments, the target gene is located on a linear chromosome. In some embodiments, the target gene is located on an ecDNA. In certain embodiments, the target gene is located on a second ecDNA. In some embodiments, the cell is a cancer cell. In some embodiments, the cell is a precancerous cell. In certain embodiments, the cell is obtained from a plurality of cells that includes the cancer cells. In some embodiments, the cell is obtained from a subject. In some embodiments, the subject is at least one of: diagnosed with, suspected of having, and at risk of having a cancer. In some embodiments, the cell is obtained from a cell culture. In certain embodiments, the cell is a vertebrate cell. In some embodiments, the cell is a mammalian cell, and optionally is a human cell.

According to another aspect of the invention, methods of determining an oncogene status of a cancer are provided, the methods including: (a) identifying in a cancer cell an oncogene modulated by an ecDNA, and (b) determining one or more of a level and an effect of the ecDNA modulation of the oncogene as a determination of the oncogene status of the cancer. In some embodiments, a means of identifying in step (a) comprises: (i) detecting an interaction between an ecDNA and one or more target genes located in DNA of the cancer cell, wherein the detecting includes directly measuring chromatin interactions between the ecDNA and a regulatory element of the one or more target genes; (ii) identifying the target genes in the interactions detected in step (i) in which the target gene's or genes' transcription is modulated by the detected interaction; and (iii) determining whether one or more of the target genes identified in step (ii) is an oncogene, wherein the determination of the target gene as an oncogene identifies the oncogene as an ecDNA-modulated oncogene in the cancer cell. In certain embodiments, the identifying in step (ii) includes measuring a level of transcription of the identified target gene and comparing the measured level to a control level of transcription of the target gene. In some embodiments, the transcription modulation is an increase in transcription. In some embodiments, a means of detecting in step (i) includes a ChIA-PET method. In some embodiments, a means of detecting in step (i) includes a Hi-C method. In certain embodiments, the regulatory element includes a promoter for the target gene. In some embodiments, activation of the promoter increases transcription of the target gene. In some embodiments, the target gene is located on a linear chromosome. In certain embodiments, the target gene is located on an ecDNA. In some embodiments, the target gene is located on a second ecDNA. In some embodiments, the cell is a cancer cell. In some embodiments, the cell is a precancerous cell. In certain embodiments, the cell is obtained from a plurality of cells that include the cancer cells. In some embodiments, the cell is obtained from a subject. In some embodiments, the subject is at least one of: diagnosed with, suspected of having, and at risk of having a cancer. In some embodiments, the cell is obtained from a cell culture. In some embodiments, the cell is a vertebrate cell. In certain embodiments, the cell is a mammalian cell, and optionally is a human cell. In some embodiments, a means for detecting the one or more of the level and the effect of the ecDNA modulation of the oncogene includes directly measuring inter-chromosomal chromatin contact frequencies between the modulating ecDNA and the modulated oncogene. In some embodiments, a means for detecting the one or more of the level and the effect of the ecDNA modulation of the oncogene includes determining a level of transcription of the one or more ecDNA-modulated oncogene, wherein the level of transcription determines an oncogene status of the cancer. In certain embodiments, the method also includes repeating steps (a) and (b) in a cancer cell obtained from a second plurality of cells comprising the cancer, and comparing one or more the level or effects detected in the cancer cell obtained from the first plurality of cells to the level or effects, respectively, detected in the cancer cell obtained from the second plurality of cells, wherein a difference in one or both of the level and effects indicates a change in the oncogene status of the cancer. In some embodiments, the method also includes contacting a candidate therapeutic agent with the second plurality of cancer cells after determining the oncogene status in the cancer of the first plurality of cancer cells and before determining the oncogene status of the second plurality of cancer cells and determining an effect of the contact with the candidate therapeutic agent on the oncogene status of the second plurality of cancer cells. In some embodiments, the first and second plurality of cells are obtained from a subject. In certain embodiments, the subject is one or more of: diagnosed with, suspected of having, and at risk of having a cancer. In some embodiments, the first and second plurality of cells are obtained from cell culture. In some embodiments, the cancer cell is a mammalian cell, optionally is a human cell. In certain embodiments, the first and second plurality of cells comprise cancer cells. In some embodiments, the method also includes assisting in selecting a treatment for the cancer, based at least in part on the determined oncogene status of the cancer. In certain embodiments, the method also includes identifying in the cancer cell one or more additional oncogenes modulated by one or more additional ecDNAs, and determining one or more of a level and an effect of the ecDNA modulation of the identified one or more additional oncogenes as a determination of the oncogene status of the cancer.

According to another aspect of the invention, methods of identifying an extrachromosomal DNA (ecDNA) in a cell, are provided, the methods including directly detecting a physical interaction between a non-linear DNA molecule and at least one linear chromosome using a chromatin interaction analysis method comprising: (a) contacting a cell with a fixative and performing chromatin proximity ligation on DNA isolated from the cell; (b) performing chromatin immunoprecipitation; (c) generating a library from the DNA immunoprecipitated in step (b); (d) sequencing the library generated in step (c) to generate sequencing data; and (e) analyzing the sequencing data generated in step (d) to detect the ecDNA. In certain embodiments, steps (a)-(c) are performed using a ChIA-PET or HI-C method. In some embodiments, step (b) comprises use of anti-RNAPII antibody in the chromatin immunoprecipitation. In some embodiments, steps (c) and (d) comprise use of paired-end tags and high throughput sequencing, respectively. In certain embodiments, the method also includes determining a frequency of the detected physical interaction. In some embodiments, the method also includes determining size of the non-linear DNA molecule. In some embodiments, the method also includes determining a number of copies of the non-linear DNA molecule in the cell. In some embodiments, the method also includes determining an average per-cell number of copies of the non-linear DNA molecule in a plurality of the cells at a first time and comparing the determined average to a control average per-cell number of copies of the non-linear DNA molecule. In certain embodiments, the control average per-cell number of copies of the non-linear DNA molecule is an average per-cell number of copies of the non-linear DNA molecule determined in the plurality of cells at a different time point. In some embodiments, the method also includes determining the sequence of at least a portion of the non-linear DNA molecule. In some embodiments, the method also includes identifying the presence of an oncogene sequence in the determined sequence. In certain embodiments, the cell is a cancer cell. In some embodiments, the cell is a precancerous cell. In some embodiments, the cell is obtained from a plurality of cells comprising cancer cells. In certain embodiments, the cell is obtained from a subject. In some embodiments, the subject is at least one of: diagnosed with, suspected of having, and at risk of having a cancer. In certain embodiments, the cell is obtained from a cell culture. In some embodiments, the cell is a vertebrate cell. In some embodiments, the cell is a mammalian cell, and optionally is a human cell. In certain embodiments, the method also includes performing steps (a)-(e) on a plurality of cells.

According to yet another aspect of the invention, methods of selecting a treatment to reduce a cancer in a subject are provide, the methods including identifying the presence of one or more specific ecDNA-modulated oncogenes in a cancer cell obtained from a subject and selecting one or more treatments based on the identified ecDNA-modulated oncogenes. In some embodiments, the identifying includes any embodiment of an aforementioned method of identifying an extrachromosomal DNA (ecDNA) in a cell, the method including: (a) detecting a chromatin interaction between a non-linear DNA molecule and at least one linear chromosome of a chromosome pair, wherein the interaction includes a contact between the non-linear DNA molecule and the at least one linear chromosome of the chromosome pair, and wherein the presence of: (i) a significantly high frequency of the detected chromatin interaction in the cell; (ii) contact between the non-linear DNA molecule and at least one linear chromosome of each of the chromosome pairs in the cell; and (iii) an increase in an average per-cell number of copies of the non-linear DNA molecule in a plurality of the cells over time, identifies the non-linear DNA molecule as an ecDNA. In some embodiments, the identifying includes any embodiment of an aforementioned method of identifying an extrachromosomal DNA (ecDNA) in a cell, the method including: directly detecting a physical interaction between a non-linear DNA molecule and at least one linear chromosome using a chromatin interaction analysis method including: (a) contacting a cell with a fixative and performing chromatin proximity ligation on DNA isolated from the cell; (b) performing chromatin immunoprecipitation; (c) generating a library from the DNA immunoprecipitated in step (b); (d) sequencing the library generated in step (c) to generate sequencing DNA; and (e) analyzing the sequencing data generated in step (d) to detect the ecDNA.

### BRIEF DESCRIPTION OF THE DRAWINGS

FIG. 1A-B provides diagrams and traces showing that ecDNA signatures can be distinguished by the distribution of trans-chromosomal interaction frequency (nsTIF) across 23 chromosomes. FIG. 1A provides Circos plots of the trans-interactions mediated by ecDNA regions across all 23 chromosomes in HF-3016 and HF-3177 ecDNA (+) cell lines. Intensive connections between ecMYC, ecEGFR and ecCDK4 regions are shown. FIG. 1B shows distribution of genome-wide normalized sum TIF (nsTIF) in 50-Kb bin size in the ecDNA (+) HF-3016 and HF-3177 cell lines as well as ecDNA (−) HF-3035 line. Elevated nsTIFs are observed on chromosomes 7, 8 and 12. Distributions of nsTIFs along the full-length chromosomes 7, 8 and 12 are shown below and regions with elevated nsTIF values are well-matched with known ecEGFR, ecMYC and ecCDK4 regions.

FIG. 2A-D shows profiles, a graph and box plots showing evidence that ecDNAs exhibit strong enhancement of H3K27ac modification of broad spans. FIG. 2A provides H3K27ac modification enrichment profiles within ±3 Kb of the chromosomal non-coding anchors interacting with the promoters of ec-oncogenes found in each of the ecDNA (+) cell line across HF-2354, HF-2927, HF-3016 and HF-3177. The numbers of anchors found in each line are labelled and each region is shown as a row ordered by the signal intensity (from the top the highest to the lowest intensity) detected in their corresponding cell line. FIG. 2B shows the concordance between the distribution of chromatin interaction frequency and H3K27ac signal density across the ecEGFR region (chr7: 54,929,292-55,441,765) in HF-2927. Lower panel: H3K27ac signal density profile of the ecEGFR region in ecDNA (+) HF-2927 (upper). For comparison, H3K27ac signal density profile from the same region in the ecDNA (−) HF-3035 line is shown (lower). Regions are highlighted to show the differences in signal intensity and span. FIG. 2C-D shows box plots showing the fold enrichment (FIG. 2C) and span size distributions (FIG. 2D) of H3K27ac peaks on the ecDNAs (Group A, n=17, 16, 96 and 70), their corresponding trans-interaction chromosomal anchors (Group B, n=166, 634, 913 and 745) and the remaining genome-wide peaks (Group C, n=38,259, 40,413, 48,751 and 56,041) from each of the four ecDNA (+) lines. In ecDNA (−) HF-3035 line, Group A (n=182) refers as the H3K27ac peaks found in the collective ecDNA equivalent regions and Group C (n=53,529) represents the remaining genome-wide peaks detected. Y-axes are in log² and log¹⁰ scales in FIG. 2C and D, respectively. Center line, median; boxes, first and third quartiles; whiskers, 1.5× the interquartile range (IQR); points, outliers. *: P value <0.005 (one-sided Wilcoxon rank-sum test). For sample sizes and exact P values for each pair-wise comparison. In each of FIG. 2C and FIG. 2D from left to right on the X axis the first A, B, and C indicate data from HF-2354, the second A, B, and C indicate data from HF-2927, the third A, B, and C indicate results from HF-3016, the fourth A, B, and C show data from HF-3177, and the final A and C show data from HF-3035.

FIG. 3A-C provides Venn diagrams, a schematic diagram and a table illustrating ecDNA-mediated trans-interacting genes and their associated interaction networks. FIG. 3A-B shows Venn diagrams that display the numbers of ecDNA-connected genes (FIG. 3A) and oncogenes (FIG. 3B) in each of the four ecDNA (+) cell lines and their overlaps. FIG. 3C illustrates process flow to define interaction anchors, nodes, hubs and community (see Methods in Examples section). Genomic regions connected by chromatin loops were defined as anchors. Non-overlapping anchors were merged as nodes with connectivity scores (numbers of connecting nodes), of which, high connectivity nodes (≥average connectivity scores+3 S.D.) were classified as hubs. Hubs and nodes with extensive connectivity were defined collectively as a community. The numbers of communities, hubs and oncogenes associated with ecDNAs are summarized for each of the ecDNA (+) lines.

FIG. 4A-B provides a summary of the ChIA-PET analysis in five GBM-derived neurosphere cell lines. FIG. 4A illustrates steady state expression level of genes amplified within the ecDNAs from ecDNA (+) GBM derived cell lines. FIG. 4B illustrates the numbers of RNAPII binding sites and long-range chromatin interactions detected in each of the five cell lines from the ChIA-PET data. The interactions are reported in three categories, the genome-wide significant cis-interactions (PET counts ≥3, P value <0.05 and FDR <0.05, see Examples section, Methods), interactions between the ecDNA regions (intra-ecDNA interactions) and between ecDNA regions and the 23 linear chromosomes (ecDNA trans-chromosomal). Only interactions with RNAPII binding sites detected at both anchors are reported.

FIG. 5A-C shows a schematic diagram, distributions, and a box display results indicating discovery of ecDNA signatures by ChIA-PET analysis. FIG. 5A is a schematic diagram showing chromatin interaction analysis used to detect genomic regions amplified in ecDNAs and their associated chromatin contacts. RNAPII ChIA-PET assays were performed to capture all RNAPII associated chromatin. EcDNAs carry actively expressed oncogenes, are not constrained by the chromatin territories and make extensive contacts with other chromosomal regions, which can be used to discover ecDNA specific signatures and characterize their co-regulated genes within the active transcription hubs. FIG. 5B shows a distribution of normalized sums of trans-interaction frequencies (nsTIFs) across 23 chromosomes in 50-Kb bin size in HF-2927 and HF-2354. In their corresponding cell lines, the distribution of normalized nsTIF of respectively chromosome 7 and chromosome 8 (shown in the zoomed-in nsTIF plots) reveal the location of the expected ecDNA to be encompassing respectively EGFR and MYC. FIG. 5B, left side shows genome-wide 2D chromatin contact heatmaps show distinct pairs of lines at regions on chromosomes 7p11 and 8q24 indicating intensive contacts with the entire genomes. FIG. 5B, right side, shows circos plots of the trans-chromosomal contact frequency across all 23 chromosomes mediated from ecEGFR and ecMYC regions are shown. FIG. 5C provides box plot displays of the normalized nsTIFs between the known ecDNA regions and chromosomal DNA regions with copy number gains ≥3 in four ecDNA (+) cell lines. From left to right, n=31, 3,423, 11, 5, 15, 82, 25, 833. nsTIFs in ecDNA regions are higher statistically than nsTIFs in regions with copy numbers gain. P values (one sided Wilcoxon rank-sum test) are 4E-22, 4.5E-4, 4.4E-10 and 7.1E-18 for HF-2354, HF-2927, HF-3016 and HF-3177, respectively. Center line, median; boxes, first and third quartiles; whiskers, 1.5× the interquartile range (IQR); points, outliers.

FIG. 6A-C provides heatmaps demonstrating ChIA-PET assay detection of chromatin topology changes reflected by the genome structural variants. FIG. 6A shows spatial chromatin topology measured by the general chromatin contacts in ChIA-PET data can be visualized by 2D contact heatmaps. Chromosome 2 heatmaps from all five GBM patient-derived neurosphere cell lines are shown. FIG. 6B provides 2D contact heatmaps of genomic regions where PTEN (upper) and CDKN2A & CDKN2B (lower) deletions in HF-2927 and HF-3035, respectively. TAD regions are demarcated by the blue lines. Deletion of the loci is represented by the loss of chromatin contacts. FIG. 6C illustrates additional structural variants of a deletion in DMD gene, a complex rearrangement in chr.3 and a double-translocations t(3; 6) visualized by the 2D heatmaps as aberrant contact patterns.

FIG. 7A-C shows heatmaps, circus plots and a schematic diagram illustrating that EcDNAs are bound by RNAPII and mediate extensive intra-extrachromosomal and trans-chromosomal interactions. FIG. 7A provides 2D contact heatmap comparison between ecDNA (+) HF-2927 and HF-2354 to ecDNA (−) HF-3035 cell lines. The profiles of cis-interaction and RNAPII binding intensity of the ecEGFR region (chr7: 54,860, 254-55,535,856) in HF-2927 relative to the non-ecDNA EGFR gene coding region in HF-3035 and the two segments of ecMYC regions (chr8: 128,032,011-128,806,493 and chr8: 129,573,241-130,968,628) in HF-2354 relative to non-ecDNA MYC coding gene in HF-3035 are shown. FIG. 7B provides Circos plot of ecDNA regions defined in HF-2927 (left) and HF-3177 (right) ecDNA (+) cell lines. From inner to outer circles: intra-ecDNA interaction loops between different regions within ecDNA, blue: the distribution of intra-ecDNA interaction frequency, green: the distribution of ecDNA-chromosomal trans-interaction frequency, orange (third ring from outside): H3K27ac fold enrichment intensity, brown (second ring from outside): RNAPII binding enrichment intensity. The signal tracks are at 1 Kb resolution. High concordance between H3K27ac signals and interaction frequency are highlighted in grey. FIG. 7C is a diagram showing genomic features (promoter, intergenic, and intragenic regions) associated with the trans-interaction anchors from ecDNAs and their chromosomal targets.

FIG. 8A-D provides Circos plots, schematic diagrams, and photomicrographs, illustrating that ecDNA regions display intense cis- and trans-interactions with strong H3K27ac enrichment. FIG. 8A shows Circos plots of ecDNA regions defined in HF-2354 (left) and HF-3016 (right) ecDNA (+) cell lines. From inner to outer circles: intra-ecDNA interaction loops between different regions of ecDNAs, blue: the distribution of intra-ec interaction frequency, green: the distribution of ecDNA-chromosomal trans-interaction frequency, orange, third ring from outside: H3K27ac fold enrichment intensity, brown, second ring from outside: RNAPII binding enrichment intensity. The signal tracks are at 1 Kb resolution. High concordance between high H3K27ac enrichment and interaction frequency are highlighted in grey. FIG. 8B shows percentages of ecDNA-connected chromosomal interaction anchors (orange) vs. anchors with no connection to ecDNA (blue) overlapped with H3K27ac peaks. FIG. 8B left shows chromosomal anchors at intragenic regions (G). FIG. 8B right shows chromosomal anchors at intergenic regions (I) (outside gene coding regions). FIG. 8C illustrates cell-specific chromosomal H3K27ac peaks interact with ecMYC promoters. Genomic locations where these broad peaks resided are shown in 10-Kb windows with starting locations chr3: 42,090,000; chr5: 148,938,000, chr1: 224,353,000, chr1: 33,906,500, chr19: 18,408,000, chr1: 207,060,000 in HF-3177; chr3: 195,902,000, chr10: 10,0120,000 in HF-3016 and chr7: 63,920,000 in HF-2354. FIG. 8D provides images showing H3K27ac immunostaining on metaphase chromosomes from HF-2927 cells. Distinct DAPI positive extrachromosomal spots overlap with H3K27ac staining spots.

FIG. 9A-E provides box plots and schematic diagrams illustrating that ecDNA-mediated chromatin interactions target oncogenes for active transcription in spatially aggregated sub- nuclear networks. FIG. 9A shows distributions of RNA expression (FPKM) between chromosomal genes trans-interacted with ecDNA (n=1,887, 1,270, 1,483 and 1,157) and genes with no trans-chromosomal interactions (n=483, 194, 653 and 597) from each of the four ecDNA (+) cell lines. * indicates P value <0.005 (one-sided Wilcoxon rank-sum test). For sample sizes and exact P values for each pair-wise comparison. FIG. 9A from left to right on the X axis the first +, − shows results for HF-2354, the second +, − shows results for HF-2927; the third +, − shows results for HF-3016; the fourth +, − shows results for HF-3177. FIG. 9B shows distributions of gene expression (FPKM) of chromosomal genes with increasing degree (0-9) of ecDNA contact frequency. For each ecDNA (+) line, 95% confidence interval of the fitted values are shaded. Smoothened FPKM is represented as the solid fitted line. FIG. 9B, each set of four boxes for each trans-interaction frequency (1-10) shows results left to right for: HF-2354, HF-2927, HF-3016, and HF-3177. FIG. 9C is a box plot showing the expression level of ecDNA-connected oncogenes (n=87, 56, 78 and 54) vs. whole transcriptome (n=21,186, 18,988, 19,206 and 19,180). P values of pair-wise comparison by one-sided Wilcoxon rank-sum test are 1.2E-7, 1.4E-5, 9.0E-8 and 1.1E-5. FIG. 9A-C, center line, median; boxes, first and third quartiles; whiskers, 1.5× the interquartile range (IQR); points, outliers. FIG. 9C from left to right on the X axis the onco, wild type (WT) shows results for HF-2354, the second onco, WT shows results for HF-2927; the third onco, WT shows results for HF-3016; the fourth onco, WT shows results for HF-3177. FIG. 9D is a diagram showing that oncogenes are clustered within spatial proximity via ecDNA-mediated chromatin interactions. Examples of two communities mediated by ecDNA connectivity hubs in HF-3016 and HF-3177 are shown. Trans-interactions among nodes are represented in tan lines with thickness represented by log¹⁰(iPET count). Blue circles are the gene (promoter) nodes, purple circles are gene (promoter) nodes annotated as oncogenes, grey circles are intergenic nodes. Size of circle is proportional to connectivity score (the number of edges in the entire genome-wide network) except the ecDNA circles which were adjusted manually to smaller sizes. Selected genes are labeled to show their variability among community. FIG. 9E is a schematic diagram of a model illustrating that ecDNAs, function as mobile enhancers, make extensive cis- and trans-chromosomal interactions to recruit oncogenes into active transcription hubs and facilitate global transcriptional amplification in cancer cells.

FIG. 10A-C provides a chart, a schematic diagram and box plots showing that ecDNA trans-connecting genes are actively transcribed. FIG. 10A illustrates high correlation between all expressed RNA measured from five GBM derived neurosphere cell lines, representing the consistency of the RNA-seq analysis. FIG. 10B illustrates that based on their ecDNA connectivity status, genes are classified in three different categories; Group I:

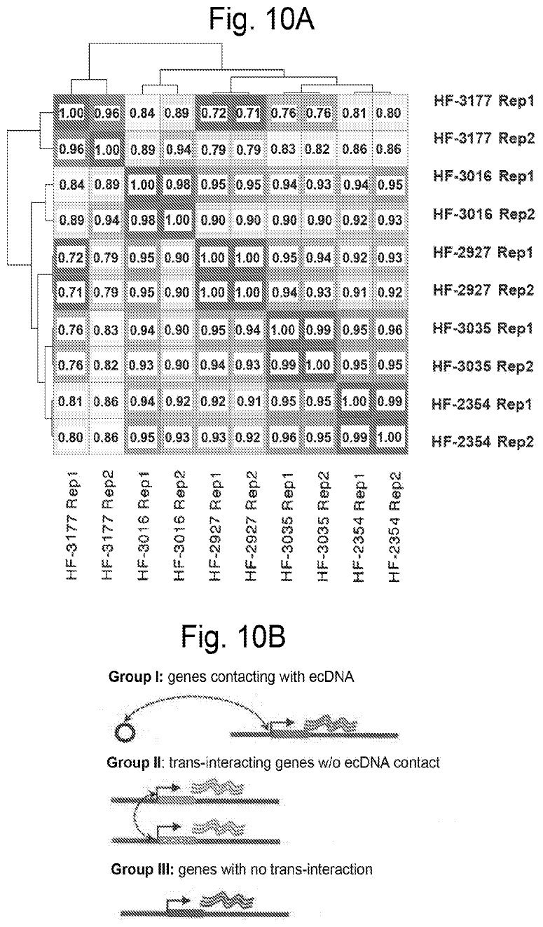

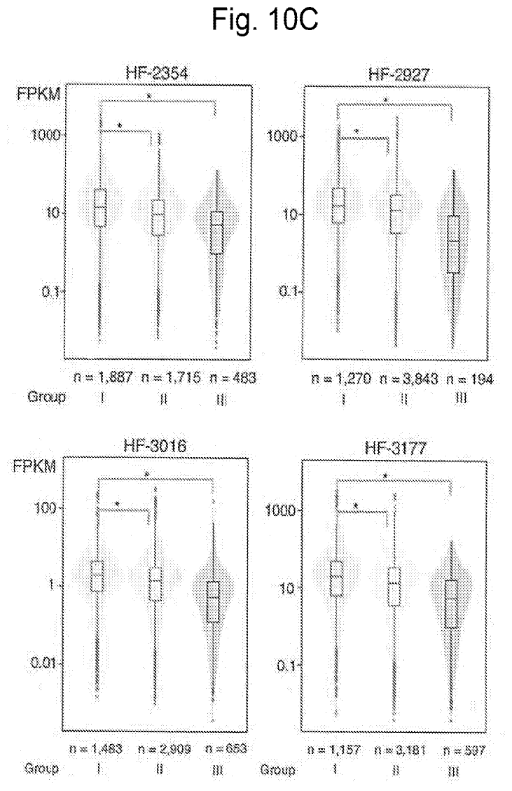

genes with promoters connecting to ecDNAs, Group II: genes with promoters trans-connect with other promoters but with no ecDNA connection, and Group III: genes with no trans-interactions. FIG. 10C provides box plots of steady-state RNA expression (FPKM) of genes from Group I, II and III in HF-2354, HF-2927, HF-3017 and HF-3177 ecDNA (+) cell lines. * represents significant P values based one-sided Wilcoxon rank-sum test. Exact P values for the pair-wise comparisons were identified and the genes classified in each of Groups I, II and III.

### DETAILED DESCRIPTION

The invention, in part, relates to methods of identifying extrachromosomal circular DNA (ecDNA) and the role of ecDNAs in diseases such as cancers. Certain methods of the invention include characterization of ecDNA and its oncogenic alteration in cancer genomes. It has now been determined that chromatin-interaction assays, such as but not limited to ChIA-PET chromatin interaction assays, can be utilized to advance the identification of ecDNAs and to characterize genome-wide ecDNA-mediated chromatin contacts that functionally impact transcriptional programs in diseases such as, but not limited to, cancers. Studies were performed, some of which are described herein, using ecDNAs in glioblastoma patient-derived neurosphere cultures. In these studies ecDNAs were identified by the presence of their widespread interchromosomal interactions. The ecDNA-chromatin contact foci were marked by the broad and high-level signals converging predominantly on chromosomal promoters, indicating a major regulatory role in genome-wide activation of chromosomal gene transcription. In some embodiments of the invention, the signals comprised H3K27ac signals. Deciphering the chromosomal targets of ecDNAs revealed an association with actively expressed oncogenes spatially clustered within ecDNA-chromatin connectivity networks. The results of studies performed indicated that ecDNAs, beyond manifestations of oncogene amplification, function as mobile transcription-amplifying elements to activate oncogene expression in cancers.

Identifying ecDNAs

To identify chromatin organization of ecDNA, and how the organization contributes to gene transcription regulation, chromatin interaction assays were utilized to examine and interrogate both general spatial chromatin organization and protein factor mediated long-range chromatin interactions, on the same cell lines. Non-limiting examples of chromatin interaction assays that may be used in some embodiments of the invention are: ChIA-PET methods, ChIP methods, and Hi-C methods. It has now been demonstrated that known ecDNAs are identifiable through their intense and aberrant intra- and inter-molecular genome-wide chromatin contacts. Also, studies performed to decipher the RNA polymerase II (RNAPII)-mediated ecDNA connectomes and their chromosomal partners, resulted in the identification of an association between ecDNA and actively expressed autosomal oncogenes. The finding indicated mechanisms of ecDNA function as mobile transcriptional enhancers that promote tumor progression.

In addition to providing a detailed characterization of ecDNA targeted chromatin interactomes in cancer genomes, the use of chromatin interaction assays as disclosed herein, provides an effective means to precisely map the amplified genomic domains within ecDNAs based on their intensive chromatin contacts within and between ecDNAs, as well as between ecDNAs and linear DNA. Prior methods used in attempts to characterize ecDNA utilized either imaging-based analysis or computational analysis of whole-genome sequencing and DNA copy number data. Embodiments of the invention disclosed herein differ from prior methods that relied on structural analysis of regions with copy number gain or microscopy imaging approaches, at least in that methods provided herein can be used to directly measure inter-chromosomal chromatin contact frequencies through chromatin interaction assays such as, but not limited to: ChIA-PET and Hi-C. Embodiments of methods of the invention provide an unbiased approach that can be used to identify one or more ecDNA signatures such as, but not limited to: ecDNA size, size comparisons between different ecDNAs, ecDNA copy numbers; ecDNA sequence information, and ecDNA sequence context. In addition, embodiments of methods of the invention can be used to determine and/or assess one or more characteristics such as, but not limited to: contact frequency and pattern between different regions of ecDNA molecules and chromosomal DNA molecules. Use of chromatin interaction assays in methods of the invention provides insight into ecDNA molecule physical structure and continuity.

Certain aspects of the invention include methods of identifying one or more ecDNAs in a cell or a plurality of cells. The methods may include a means of detecting a chromatin interaction between a non-linear DNA molecule and at least one linear chromosome. In some embodiments of the invention, the method comprises detecting a chromatin interaction between a non-linear DNA molecule and at least one linear chromosome of a chromosome pair. The term “detecting a chromatin interaction” as used herein means detecting one or more of a frequency of a chromatin interaction, an ecDNA in the interaction, a target gene in the interaction, and other characteristics of the chromatin interaction. Characteristics of the chromatin interaction may include, but are not limited to one or more of: a size of the non-linear DNA, and a number of copies of the non-linear DNA molecule in the cell, etc. In some embodiments, methods of the invention include comparing one or more characteristics of chromatin interaction, for example, determining an average per-cell number of copies of the non-linear DNA molecule in a plurality of the cells at a first determination time and comparing the determined average to a control average per-cell number of copies of the non-linear DNA. In some embodiments of the invention, comparing one or more characteristics of a chromatin interaction may include determining an average per-cell number of copies of the non-linear DNA molecule in a plurality of the cells at a first determination time and comparing the determined average to a control average per-cell number of copies of the non-linear DNA. It will be understood that in some embodiments of the invention, a control average per-cell number of copies of the non-linear DNA molecule is an average per-cell number of copies of the non-linear DNA molecule determined in the plurality of cells at a different time point than the time point of another determination. Other non-limiting examples of detecting a characteristic of a chromatin interaction include: determining the sequence of at least a portion of the non-linear DNA, and identifying the presence an oncogene sequence in the determined sequence.

Particular features have now been identified that can be used to identify an ecDNA in a cell. The features are identified by one or more determined characteristics of chromatin interactions in a cell. For example, identifying a chromatin interaction as comprising a contact between the an ecDNA and at least one linear chromosome includes, identifying (i) a significantly high frequency of the detected chromatin interaction in the cell; (ii) contact between the non-linear DNA molecule and at least one linear chromosome, (for example, but not limited to, at least one linear chromosome of each of the chromosome pairs in the cell); and (iii) an increase in an average per-cell number of copies of the non-linear DNA molecule in a plurality of the cells over time. In some embodiments of the invention, the presence of these three features (features i-iii) identifies the non-linear DNA molecule as an ecDNA.

Several characteristics of ecDNA have now been identified and in some embodiments of methods of the invention the presence of the characteristics in a non-linear DNA molecule in a cell confirms the identity of the non-linear DNA molecule as ecDNA. Certain embodiments of methods of the invention include determining 1, 2, or 3 of the following characteristics as a means with which to confirm the identification of a detected non-linear DNA as an ecDNA. One such characteristic of ecDNA that has now been identified is that chromatin interactions that include an ecDNA and its target gene(s) occur at a significantly higher level and frequency than other types of chromatin interactions in cells. As used herein the term “significantly high frequency” of the detected chromatin interaction means the number of such chromatin interactions detected is statistically significantly higher than the number of chromatin interaction detected if the chromatin interactions do not include an ecDNA.

Another characteristic of ecDNA that has now been identified is the presence of contact between an ecDNA and at least one linear chromosome in a cell. In some embodiments of the invention, the ecDNA characteristic comprises the presence of contact between an ecDNA and at least one linear chromosome in each chromosome pair in the cell. For example, though not intended to be limiting, a non-linear DNA molecule that is identified as having contact with at least one chromosome in a cell is identified as an ecDNA. In another non-limiting example, a non-linear DNA molecule that is identified as having contact with at least one chromosome in each of the chromosomes in the cell (e.g., in each of the 23 pairs of chromosomes in a human diploid cell), is identified as an ecDNA. In the latter example, there would be an ecDNA interaction with a gene target located on least one linear chromosome in each of the 23 chromosome pairs in the human cell.

A third characteristic of ecDNA that has now been identified is that over time, the average per-cell number of copies of an ecDNA increases. Thus, as a non-limiting example, in a cell sample obtained from population of cells, an average of the number of copies of a non-linear DNA is determined. At a later time, a second cell sample is obtained from the population of cells and an average of the number of copies of the non-linear DNA is determined and compared to the average number determined in the first sample. An increase in the average number of the non-linear DNA in the later sample, supports a conclusion that the non-linear DNA is ecDNA. In some embodiments of the invention, an increase in the average number may be an increase of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 250%, or 500%, including all percentages within the stated range. In some embodiments of the invention, an increase in the average number may be an increase of at least 500%, 1000%, 1500%, 2000%, or 5000%.

In some embodiments of the invention a cell or plurality of cells is obtained from a sample, culture, or subject at two or more different time points. The length of time between obtaining two cell samples may be independently selected based on factors including but not limited to: convenience for a subject, convenience for health care provider, status or stage of a cancer, rate of development of a cancer, tumor growth rate, etc. In some embodiments of the invention an interval of time between obtaining two cell samples is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 days. In some embodiments of the invention an interval of time between obtaining two cell samples is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, and 52 weeks. In some embodiments of the invention an interval of time between obtaining two cell samples is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments of the invention an interval of time between obtaining two cell samples is at least 1, 2, 3, 4, 5, 6, 7, 8, 9, 10, or more years. It will be understood that more than two cell samples may be collected for use in certain embodiments of methods of the invention and that a time interval between obtaining any two samples may be independently selected and can, but need not be identical to other time intervals at which cell samples are obtained.

ecDNAs and Target Oncogenes

Work described herein demonstrates regulatory relationships between ecDNA and target genes, a non-limiting example of which is an oncogene. As used herein the term “target oncogene” means an oncogene whose activity is modulated by an ecDNA. An ecDNA that modulates transcription of a target oncogene may do so via a gene regulatory system (GRS). A GRS includes a gene regulator in the ecDNA. The ecDNA gene regulator may be a gene activator, or in some instances may be a gene silencer. It will be understood that an interaction between an ecDNA and its target gene may include binding of an ecDNA gene regulator sequence with a transcription factor, which also binds a gene regulatory element of the target oncogene. The binding of the transcription factor with the gene regulatory element, which comprises a specific short region of DNA, stimulates transcription of the target oncogene.

A transcription factor that binds the ecDNA gene regulator and the target oncogene may comprise a complex of polypeptides and acts as a “connector” between the ecDNA gene regulator and a gene regulatory element of the target oncogene. The term “gene regulatory element” means a DNA sequence, such as but not limited to a promoter or enhancer sequence, that is responsible for and/or involved in transcription of the target oncogene. As used herein an interaction between an ecDNA gene regulator and its target oncogene comprises contact of the ecDNA gene regulator with a transcription factor, which also contacts a gene regulatory element for the target oncogene, for example a promoter sequence of the target oncogene. The ecDNA/oncogene interaction enhances transcription of the target oncogene, which may result in or promote development of a cancer. In some embodiments, a cancer may be in a cell comprising the ecDNA and its target oncogene. A cancer cell resulting from an ecDNA/oncogene interaction may be in a subject. Different target oncogenes may have enhanced transcription due to an ecDNA/oncogene interaction and a cell may include one, two, or more different oncogenes whose transcription is increased by an ecDNA/oncogene interaction. Two or more cells in a subject may include the same target oncogenes or may include different target oncogenes whose transcription is modulated by one or more ecDNAs. A cancer in a subject may arise and/or be maintained by the activity of one, two, or more different oncogenes, with the transcription of each modulated by one or more ecDNAs.

In instances in which two or more different oncogenes are activated in a cancer in a subject, two or more different cancer therapeutics that are directed to different oncogenes and/or different ecDNA/oncogene interactions may be used to effectively treat the cancer in a subject. In some embodiments of the invention, a cancer therapeutic may be selected and/or administered to a subject based at least in part on the presence or absence of an interaction between an ecDNA and a specific oncogene and the modulation of that oncogene by the interaction.

In addition to methods of the invention that can be used to identify ecDNAs, certain methods of the invention can be used to assess the status of a cancer in a cell by assessing the presence or absence of an interaction between an ecDNA and its target oncogene. These methods are based, in part, on an improved understanding of 3-dimensional genome organization and its role in gene regulation. Certain embodiments of the invention comprise methods of identifying an interaction between an ecDNA and at least one target oncogene in a cell. In addition, certain embodiments of methods of the invention can be used to assess the frequency and effect of ecDNA/oncogene interactions. In some embodiments of the invention interaction of an ecDNA with its target oncogene enhances (also referred to herein in as “increases”) transcription of the target oncogene.

### Chromatin Interaction Assays

Certain aspects of the invention include use of chromatin interaction assays. In certain embodiments of methods of the invention, chromatin interaction assays are used to determine structural features of chromosomes, to identify ecDNA in cells, and to determine interactions between ecDNA and target genes, such as, but not limited to, oncogenes. Chromatin interaction assays and analysis as disclosed herein also permit determination of transcriptional regulation of target oncogenes by ecDNA.

A non-limiting example of a means with which to assay chromatin interactions in cells is Chromatin Interaction Analysis by Paired-End Tag Sequencing, (ChIA-PET), which combines ChIP with chromatin conformation capture (3C) technology (see Fullwood, et al. 2009, Nature, Vol. 426 (7269):58-64; the contents of which is incorporated herein by reference in its entirety.) ChIA-PET methods permit detection of interactions between distant DNA regions that interaction with each other via a protein or protein complex of interest. In a non-limiting example of a ChIA-PET method, chromatin from a cell is cross-linked, digested, and pulled down using an antibody against a protein of interest. Linker sequences are ligated to the ends of the DNA and the presence of the linker sequences facilitate their ligation to each other (Zhang et al., 2012, Methods Vol. 58, No. 3:289-299; the contents of which is incorporated herein by reference in its entirety). This results in hybrid DNA fragments from two different regions of the genome. The resulting library is sequenced and the results identify DNA regions that interact with each other and the protein of interest. ChIA-PET has previously been used to map the interactions of transcription factors, and its use in embodiments of methods of the invention permit identification of ecDNA, target genes of ecDNAs, and the modulation of transcription of the target genes by ecDNA/target gene interactions. Chromatin interaction analysis using ChIA-PET has now been used for genome-wide discovery of chromatin interactions. Prior visual and computational methods were generally unsuitable to detect weak or dynamic interactions and this drawback is remedied through use of ChIA-PET methods.

Another non-limiting example of a means of identifying and assessing chromatin interactions that is used in certain embodiments of the invention is a Hi-C assessment method. Hi-C-based methods may be used in embodiments of the invention due in part to its ability to provide unbiased genome-wide coverage able to measure chromatin interaction intensities between any two given genomic loci. In certain embodiments of the invention, Hi-C data can be used to assess genome-wide chromatin organization, such as topologically associating domains (TADs), linearly contiguous regions of the genome that are associated in 3-D space. Various art-known algorithms are routinely used to identify TADs from Hi-C data (see for example, Dixon et al, 2012 Nature 485 (7398):376-80; the contents of which is incorporated herein by reference in its entirety).

The following is a general overview of elements in Hi-C analysis. A genome of a cell is cross-linked, which retains the interactions between genomic loci. There are a number of art-known fixation methods suitable for use to cross-link the cell genomes in Hi-C methods. After the cross-linking, the cross-linked genome is cut using a restriction enzyme, with the size of the resulting fragments determining the resolution of interaction mapping by the Hi-C method. Non-limiting examples of restriction enzymes that may be used are those that make cuts every 4000 bp, such as EcoR1 or HindIII, resulting in ˜1 million fragments in the human genome. For higher resolution interaction mapping, restriction enzymes that cut more frequently may also be used. Following the digestion step the pieces are randomly ligated under conditions that that favor ligation between cross-linked interacting fragments instead of ligation between fragments that are not cross-linked. The interacting loci can then be quantified by amplifying ligated junctions using a method such as, but not limited to polymerase chain reaction (PCR), see for example, Naumova, et al., 2012 Methods. 58 (3):192-203 and Gavrilov, et al., 2013 PLOS One. 8 (3):e60403, the contents of each of which is incorporated herein by reference in its entirety.) Certain Hi-C methods may include high-throughput sequencing to find the nucleotide sequence of fragments, for example a sequence of an ecDNA.

Additional steps and procedures that may be included in a ChIA-PET and/or HI-C method used in an embodiment of the invention are described herein. For example, computational methods, analysis methods, data assessment methods, etc. In addition to ChIA-PET and HI-C related methods of chromatin interaction analysis other chromatin interaction assays and analysis methods are known and used in the art and are suitable for use in embodiments of methods of the invention. Non-limiting examples of additional chromatin interaction assay/analysis methods include: 4C (Nat Genet. 2006 November; 38(11):1348-54), Hi-C (Lieberman-Aiden, E. et al. 326, 289-293 (2009), capture Hi-C (Nat Genet. 2015 June; 47(6):598-606) PLAC-seq (Cell research. 2016; 26(12):1345-8) and HiChIP (Nat Methods. 2016 November; 13(11):919-922); the contents of each of which is incorporated by reference herein in its entirety.

### Interaction Analysis—General Information and Non-Limiting Embodiments

It will be understood that the following descriptions of interaction analyses are not intended to be limiting but intended to illustrate results and information obtained using embodiments of methods of the invention. Other specific cell types, regions, oncogenes, etc. can be used in embodiments of the invention. Additional information regarding analysis of interactions between ecDNAs and their target oncogenes is set forth in the Examples section herein. Such information includes but is not limited to: demonstrated use of embodiments of methods of the invention for discovery of known and unknown ecDNA regions using ChIA-PET data, ChIP-seq library construction and data analysis, etc.

Certain embodiments of methods of the invention comprised quantification of a degree of chromatin contacts. In a non-limiting example, quantification is performed using a metric that described the genome-wide trans-interaction frequencies (nsTIF) normalized across all 23 chromosomes. EcDNA regions were identified as having highly elevated nsTIF levels in regions with ecDNA. In addition, high nsTIF regions that linked to ecDNA segments show trans contacts across the entire genome, indicating a dynamic ecDNA connectivity from the extrachromosomal genetic elements. The observed genome-wide contact pattern in this example, results from the mobile nature of ecDNA. In this non-limiting example, a verification step was performed to verify that the identified high nsTIFs were specific to the extrachromosomal nature of the ecDNAs. In this example, the results confirmed that the elevated contact frequency of ecDNA across the genome was not the result of a DNA dosage effect alone but was determined by its autonomous capacity.

In another non-limiting example, a high frequency of cis-interactions was detected within the genomic regions of ecDNAs. In HF-2927 ecDNA (+) cells, the cis-interaction intensity observed within the ˜530 Kb ecDNA region was 2,879, a 240-fold increase, compared to only 12 in the same region in ecDNA (−) HF-3035 cells. The high contact increase directly reflected both size and genomic structure of this ecEGFR. Similarly, the results indicated intensive cis-interactions within and between the two segments of the defined ecMYC region in HF-2354. Extensive RNAPII tethered chromatin contacts (defined as RNAPII binding detected at DNA regions connected by the interactions, referred as anchors) were detected both in cis between different regions within ecDNA (referred as intra-ecDNA) and in trans (referred as trans-interactions) with other genes or regulatory elements on linear chromosomes. It was identified that the intra-extrachromosomal connectivity patterns show distinct pairs of loops with high frequency interactions and foci of intense contacts, which were expected to be collectively derived from contacts between different ecDNA molecules and folding within individual ecDNAs. Among the trans-interactions between ecDNAs and their chromosomal partners, the anchors on ecDNAs were identified as primarily in intra- or inter-genic non-coding regions and their trans-interaction chromosomal anchors were primarily localized at promoters. This juxtaposition of the interactions supports a transcriptional function for these contacts.

In a non-limiting example of assessing ecDNA interactions with transcriptional regulatory regions, H3K27ac profiling is performed to mark active enhancers and promoters. Regulation of the oncogenes amplified on ecDNA is examined by evaluating their trans-chromosomal interaction regions. These ecDNA-connecting non-coding chromosomal anchors exhibited high overlap with H3K27ac peaks; which was significantly higher than those from the trans-interaction non-coding chromosomal to anchors with no ecDNA contact supporting a conclusion that the transcription of the oncogenes on ecDNA is further enhanced by engaging enhancers on the linear chromosomes through chromatin contacts.

In certain embodiments of the invention, ecDNA interactions were assessed by observing co-occurrence between high frequency contact foci and H3K27ac peaks within the ecDNAs, results supporting a conclusion that these interaction anchors behave like active enhancers. In this non-limiting example of interaction assessment methods, H3K27ac peaks within the 530 Kb ecEGFR region co-aligned with the regions of high interaction frequency in HF-2927, and exhibited the pattern as clusters in close proximity with broader genomic spans when compared to the H3K27ac peaks in the chromosomal EGFR region in the ecDNA (−) cells (HF-3035), supporting a conclusion that enhancer signals accumulated on the chromatin contact sites of ecDNA. In this example, immunostaining using an antibody targeting H3K27ac on metaphase HF2927 cells demonstrated overlapping signals between H3K27ac and DAPI signals marking ecDNA, which confirmed the association between enhancer function and ecDNA.

In certain embodiments of the invention, methods include quantitatively assessing increases in H3K27ac signal associated with ecDNA mediated trans-chromatin interactions and it was found that H3K27ac peaks associated with ecDNA chromatin interaction anchors have significantly higher enrichment compared to those of genome-wide H3K27ac peaks with no ecDNA contacts. In this non-limiting example of an assessment, the strong enhancement of H3K27ac signal was confirmed as specific to the ecDNAs.

In another non-limiting example of use of a method of the invention, methods were used to evaluate the enhancer signatures observed in the ecDNA reminiscent of “super-enhancers”. In this non-limiting example, examinations were performed of the span sizes of the H3K27ac peaks detected in ecDNAs and their trans-interacting chromosomal anchors. It was found that H3K27ac peaks on ecDNA had significantly longer spans than the chromosomal H3K27 peaks with no ecDNA contact. Sequence analysis showed an enrichment of binding motifs of transcription factors critical in regulating RNAPII general transcription and cell proliferation, including JUN, FOS and ATF. Collectively, both cis and trans convergence of RNAPII signal with strong enhancement of H3K27ac signal supports a conclusion that ecDNA molecules are able to connect the RNA polymerase machinery broadly across the genome, corroborating a function as a genome-wide transcriptional amplifier.

In a non-limiting example, methods of the invention are used to determine whether the increase in enhancer signals associated with ecDNA trans-interactions results in active transcription. In this example, RNA expression was examined and results indicated that ecDNA interacting genes had significantly higher levels of expression compared to genes with either no other trans-chromosomal contact or genes with no contact with ecDNAs but having trans-chromosomal interactions with other genes. Furthermore, in this example method, the expression level of the ecDNA-connecting genes was identified as positively correlated with the frequency of their ecDNA contacts (measured by the numbers of independent trans-interactions), supporting a finding that ecDNA connectivity was highly associated with transcriptional activity, and a highly enhanced H3K27ac signature, suggesting that ecDNA can act as a global transcriptional amplification machinery.

Beyond the recruitment of individual oncogenes, ecDNAs were identified as the focal points where many oncogenes are brought together into spatial proximity via their interactions with ecDNAs. Many of these ecDNA-connected oncogenes reside within each of the chromatin networks, supporting a conclusion that the co-aggregation of oncogenes is a structure-based mechanism adopted by ecDNAs to achieve coordinated transcriptional co-activation to promote tumorigenesis.

### Assessing Cancer Status

It has now been shown that ecDNAs can enhance extrachromosomal and chromosomal gene transcription through chromatin interactions. Embodiments of the invention include methods to identify ecDNAs in cells. Certain embodiments of the invention provide methods with which to determining an effect of an ecDNA on a target gene, for example an oncogene whose transcription is modulated by one or more ecDNAs. It has been identified that ecDNAs can enhance expression of extrachromosomal and chromosomal gene transcription through chromatin interactions. This finding combined with the prevalence and diversity of ecDNA, identify ecDNA, an ecDNA/target oncogene interaction, and a target gene as targets for therapeutic intervention in a disease such as a cancer. Methods of the invention are based, in part, on the identification of an interplay between genetic structure and the epigenetic consequences in tumor evolution. Embodiments of methods of the invention provide a means to identify ecDNAs, to identify ecDNA interactions with a target gene, and effects of an ecDNA interaction with a target gene. The role of ecDNA activity in cancers and the unique genomic dynamics of this extrachromosomal structure, provide new approaches for targeting ecDNA and their activated chromosomal target genes and activated ecDNA target genes, for example for use in therapeutic applications.

Gene expression programs that establish and are responsible for the status or state of a cell include but are not limited to an activity of one or more transcription factors that bind an ecDNA gene regulator and a gene regulator element of a target oncogene. Non-limiting examples of specific genomic elements are enhancer elements, which bind transcription factors and can loop long distances to contact and regulate specific genes. Although the interaction between an ecDNA gene regulator sequence and a gene regulatory element such as, but not limited to a promoter of a target oncogene, has now been studied with respect to interactions between ecDNAs and gene regulatory elements on linear chromosomes. Certain aspects of the invention can be used to obtain information on the identity of extrachromosomal DNAs (ecDNAs) that are involved in cellular gene regulatory processes through their interactions with linear genomic elements, such as oncogene promoters. In addition, certain methods of the invention can be used to assess regulatory interactions between two or more ecDNAs that participate in a cell's gene expression program, including in some instances, aberrant gene expression programs such as those present in cancer cells.

### Identifying Candidate Therapeutic Agents and Selecting Treatments for Cancer

In some aspects of the invention methods are provided to identify a status of one or more oncogenes in a cancer cell. Methods of the invention can be used determine a level of transcription of one or more target oncogenes, wherein elevated levels of the one or more target oncogenes identify a likelihood of cancer. In some embodiments of the invention, a plurality of cancer cells can be a source from which to obtain cells for use in comparative studies and to test candidate treatments. For example, though not intended to be limiting, a plurality of cancer cells may be cancer cells in culture or in a subject and may be maintained in the same environment. In some embodiments of the invention one or more cancer cells from such a culture or subject are included in a method of the invention to assess the cells' status with respect to ecDNA/oncogene interactions. A different one or more of the cancer cells are contacted with a therapeutic agent or candidate therapeutic agent and the contacted cells are included in a method of the invention to assess the cells' status with respect to ecDNA/oncogene interactions. The ecDNA/oncogene interactions determined in the non-contacted and contacted cancer cells can be determined and compared to each other or to an appropriate control, providing to information on the effect of the therapeutic or candidate therapeutic on the ecDNA/oncogene interaction and the status of the cancer.

As used herein the term “status” when used in reference to a cancer cells, means the presence or absence of one or more specific ecDNA/oncogene interactions. For example, though not intended to be limiting, an initial status of a cancer may include interactions between ecDNA with oncogenes A and B, and as the cancer progresses, its status may be determined to include interactions between ecDNA with oncogenes A, B, and C.

In some embodiments of the invention, identifying an oncogene that is modulated by an ecDNA provides information that can be used to aid in selecting a treatment for a subject with a cancer. In some embodiments, a subject may be screened for a pre-disposition of cancer or to stage a cancer that is present or suspected of being present in the subject. Embodiments of methods of the invention may be used to screen for a cancer or cancer status in a subject and such methods may include one or more of: identifying an ecDNA/oncogene interaction in a cell and determining the modulating effect of the ecDNA on the target oncogene. Such methods can be used to identify the status of a cancer in a subject. In addition, methods of the invention may be used to assess an effect of a candidate agent on the modulating effect of an ecDNA on its target oncogene in a cancer, and the results of the assessment may be used to assist in selecting a treatment for the cancer.

Embodiments of methods of the invention can be used to assess a status of a cancer in one or more of a cell, tissue, subject, and plurality (or population) of cells. As used herein, the term “cancer” is used in reference to a malignant neoplasm. Exemplary cancers include, but are not limited to, acoustic neuroma; adenocarcinoma; adrenal gland cancer; anal cancer; angiosarcoma; appendix cancer; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); cervical cancer (e.g., cervical adenocarcinoma); colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal cancer; head and neck cancer (e.g., head and neck squamous cell carcinoma, oral cancer), throat cancer; hematopoietic cancers (e.g., leukemia such as acute lymphocytic leukemia (ALL); lymphoma such as Hodgkin lymphoma (HL) and non-Hodgkin lymphoma (NHL); multiple myeloma (MM); hemangioblastoma; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); malignant mesothelioma; muscle cancer; myeloproliferative disorder (MPD); neuroblastoma; neurofibroma; neuroendocrine cancer; osteosarcoma; ovarian cancer; papillary adenocarcinoma; pancreatic cancer; penile cancer; prostate cancer; rectal cancer; rhabdomyosarcoma; salivary gland cancer; skin cancer; melanoma; small bowel cancer; soft tissue sarcoma; sebaceous gland carcinoma; small intestine cancer; sweat gland carcinoma; synovioma; testicular cancer; thyroid cancer; urethral cancer; vaginal cancer; and vulvar cancer.

A cancer may be a primary cancer or a metastatic cancer, and may be considered an early or late stage cancer, or a cancer stage in a subject may be characterized with one or more cancer staging classifications known and routinely practice in the art. In some aspects of the invention a cancer is a first cancer in a subject and in certain aspects of the invention a cancer may be a relapse or recurrence of a prior cancer. In some instances, an embodiment of a method of the invention may be used to assess a status of a cancer in a subject who has not been treated with a cancer therapeutic. In certain embodiments, a method of the invention is used to assess a status of a cancer in a subject who has been or is currently being treated with one or more cancer therapeutics. Non-limiting examples of cancer therapeutics include: surgery, radiotherapy, chemotherapy, immunotherapy, dietary treatment, or other art-known therapeutic approach.

Certain embodiments of the invention include methods to assist in determining and/or selecting one or more therapeutic protocols for a subject. For example, though not intended to be limiting, some embodiments of the invention may be used to assist in selecting a treatment for a cancer in a subject based, at least in part, on the status of ecDNA/oncogene interactions identified in a cancer cell obtained from the subject. Determining the status of a cancer in a subject using an embodiment of a method of the invention, permits selection of one or more therapeutics based on the identified ecDNA/oncogene interactions. For example, though not intended to be limiting, a method of the invention can be used to detect the status of a cancer in a subject through the identification of one or more ecDNA/target oncogene interactions in a cancer cell obtained from the subject. Methods of the invention may also be used to identify one or more specific oncogenes and other components in the detected ecDNA/interactions and this information can be used to assist in selecting a treatment for the cancer in the subject. For example, if interactions between ecDNA and oncogene A and oncogene B are detected in a cancer cell from a subject, the information can assist in selecting a treatment for the cancer that results in one or more of: (i) reduces the interaction of an ecDNA with oncogene A and (ii) reduces the interaction of an ecDNA with oncogene B. Based on ecDNA/oncogene interaction information determined using an embodiment of a method of the invention, a cancer in a subject can be categorized by specific oncogene/ecDNA interactions and an appropriate treatment may be selected and administered to the subject to reduce the specific oncogene/ecDNA interaction.

In certain embodiments of the invention methods are provided that permit determining an efficacy of a cancer therapeutic administered to a cancer cell or to a subject having a cancer, suspected of having a cancer, or at increased risk of having a cancer. In a non-limiting example, an embodiment of a method of the invention is used to determine an initial status of a cancer in a cancer cell obtained from a subject. The cancer status is determined to include identified interactions between one or more ecDNAs and oncogene A, oncogene B, and oncogene C. A cancer treatment is selected for the subject based at least in part on the identified interactions of the ecDNA with the three oncogenes. Following administration of a selected treatment to the subject, methods of the invention are used for a subsequent status determination of a cancer cell obtained from the subject following the treatment. The status of the cancer determined in a cancer cell obtained before, the status of the cancer determined in a cancer cell obtained after administration of the treatment can indicate the efficacy of the treatment on the cancer in the subject. For example, a finding in a cancer cell obtained from the subject after the treatment of an interaction between an ecDNA and oncogene A but no indication of an interaction between an ecDNA and oncogene B or oncogene C, supports the efficacy of the cancer treatment in the subject and can confirm the efficacy of the treatment to reduce an enhancing interaction between the ecDNA and oncogene B and oncogene C.

Non-limiting examples of oncogenes activated via ecDNA are: epidermal growth factor receptor (EGFR), mouse double minute 2 (MDM2), cyclin-dependent kinase 4 (CDK4) and cMYC. Agents that can be used to treat cancers in which specific oncogenes are activated include, but are not limited to: EGFR inhibitors, MDM2 inhibitors, CDK4 inhibitors and cMYC inhibitors. In a non-limiting example a cancer identified using a method of the invention as comprising EGFR amplification by an ecDNA interaction with the EGFR oncogene can be treated by administration of tyrosine kinase inhibitors (TKIs) drugs to a subject having the cancer. Non-limiting examples of TKIs are: gefitinib and erlotinib. In another non-limiting example, a cancer identified using a method of the invention, as comprising CDK4 transcription increase resulting from interaction of an ecDNA interaction with the CDK4 oncogene can be treated by administration of one or both of palbociclib and ribociclib. Based on the teaching presented herein, a skilled artisan will be able to select other art-known therapeutics based at least in part aided by identification of one or more interactions between an ecDNA and an oncogene that results in increased transcription of the oncogene.

A non-limiting example of a treatment for a cancer may include administering to a subject diagnosed with a cancer, at increased risk of having a cancer, or believed to have a cancer an effective amount of an agent that interferes with and reduces an interaction between an ecDNA and a target oncogene of the ecDNA. In an ecDNA gene regulatory system (GRS) in a cell, an ecDNA includes a sequence that may be referred to as a “gene regulator” sequence, non-limiting examples of which are gene actuator sequences and gene silencer sequences in the ecDNA. A GRS that includes the ecDNA also includes a “transcription factor”, which is a complex that serves as a “contact” between the ecDNA and its target oncogene. A transcription factor may comprise a complex of polypeptides that link the ecDNA gene regulator with the ecDNA's target oncogene, through binding of the transcription factor with a “gene regulator element” of the target oncogene. For example, though not intended to be limiting, a promoter that controls transcription of the target oncogene. In some embodiments, methods of the invention include identifying a candidate target present in the interaction between the ecDNA and its target oncogene, wherein disruption of the identified candidate target disrupts the interaction between the ecDNA and its target oncogene and reduces the ecDNA enhancement of transcription of its target oncogene. As a non-limiting example, a polypeptide in a complex of a transcription factor may be an identified as a candidate target that when contacted with a therapeutic agent that disrupts the GRS, reduces the ecDNA's enhancement of transcription of the target oncogene.

In some embodiments of the invention, methods include contacting a cancer cell and/or administering to a subject a therapeutic agent that disrupts a candidate target in a GRS and reduces an interaction between the ecDNA and its target oncogene thereby reducing the ecDNA enhancement of transcription of its target oncogene. In some embodiments of the invention, a candidate target comprises the gene actuator on the ecDNA. As used herein the terms “gene actuator” and “gene enhancer” may be used interchangeably in reference to a gene regulator. In some aspects of the invention a candidate target is one or more components of a transcription factor. In some embodiments of the invention a candidate target is a gene regulator element, for example, but not limited to a promoter element for the ecDNA's target oncogene. Thus, in certain embodiments of the invention one or more of: an ecDNA activator, a transcription factor, and a gene regulator element may be identified as candidate targets against which to direct one or more therapeutic agents to treat the cancer.

In some embodiments of the invention a therapeutic agent may be administered in combination with a second therapeutic agent. In some embodiments, an agent is administered in combination with a cancer therapeutic agent or in combination with another cancer treatment such as but not limited to one or more of: radiotherapy, chemotherapy, surgery, etc. g., before, after, or interspersed with doses or administration of the cancer therapeutic agent. In some embodiments, an agent of the present invention is administered to a subject undergoing conventional chemotherapy and/or radiotherapy. In some embodiments the cancer therapeutic agent is a chemotherapeutic agent. In some embodiments the cancer therapeutic agent is an immunotherapeutic agent. In some embodiments the cancer therapeutic agent is a radiotherapeutic agent.

### Cells

It will be understood that a cell included in a method of the invention may be one of a plurality of cells. As used herein the term, “plurality” of cells may mean a population of cells. A plurality of cells may be all of the same type and/or may all have the same disease or condition. As a non-limiting example, a cell may be obtained from a population of liver cells, and other cells obtained from this population of cells will also be liver cells.

In some embodiments of the invention, a plurality of cells may be a mixed population of cells, meaning all cell are not of the same type. In another non-limiting example, a cell may be a cancer cell obtained from a plurality of cancer cells. A cell used in an embodiment of a method of the invention may be one or more of: a single cell, an isolated cell, a cell that is one of a plurality of cells, a cell that is one in a network of two or more interconnected cells, a cell that is one of two or more cells that are in physical contact with each other, etc.

In some aspects of the invention a cell may be obtained from a living animal, e.g., a mammal, or may be an isolated cell. An isolated cell may be a primary cell, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation in culture (immortalized cells). In some embodiments of the invention, a cell is a somatic cell. Somatic cells may be obtained from an individual, e.g., a human, and cultured according to standard cell culture protocols known to those of ordinary skill in the art. Cells may be obtained from surgical specimens, tissue or cell biopsies, etc. Cells may be obtained from any organ or tissue of interest, including but not limited to: skin, lung, cartilage, brain, breast, blood, blood vessel (e.g., artery or vein), fat, pancreas, liver, muscle, gastrointestinal tract, heart, bladder, kidney, urethra, and prostate gland. In some embodiments of the invention a cell is a HF-3035 cell, or an HF-2354 cell.

In some embodiments, a cell used in conjunction with the invention may be a healthy normal cell, which is not known to have a disease, disorder or abnormal condition. In some embodiments, a host cell used in conjunction with methods and compositions of the invention is an abnormal cell, for example, a cell obtained from a subject diagnosed as having a disorder, disease, or condition, including, but not limited to a degenerative cell, a neurological disease-bearing cell, a cell model of a disease or condition, an injured cell, etc. In some embodiments of the invention, a cell may be a control cell. In some aspects of the invention a host cell can be a model cell for a disease or condition.

A cell that may be used in certain embodiments of the invention is a human cell. Non-limiting examples of a cell that may be used in an embodiment of a method of the invention are one or more of: eukaryotic cells, vertebrate cells, which in some embodiments of the invention may be mammalian cells. A non-limiting example of cells that may be used in methods of the invention are: vertebrate cells, invertebrate cells, and non-human primate cells. Additional, non-limiting examples of cells that may be used in an embodiment of a method of the invention are one or more of: rodent cells, dog cells, cat cells, avian cells, fish cells, cells obtained from a wild animal, cells obtained from a domesticated animal, and other suitable cell of interest. In some embodiments a cell is an embryonic stem cell or embryonic stem cell-like cell. In some embodiments the cell is a neuronal cell, a glial cell, or other type of central nervous system (CNS) or peripheral nervous system (PNS) cell. In some embodiments the cell is an astrocyte cell. In some embodiments of the invention a cell is a natural cell and in certain embodiments of the invention a cell is an engineered cell.

Cells useful in embodiments of methods of the invention may be maintained in cell culture following their isolation. Cells may be genetically modified or not genetically modified in various embodiments of the invention. Cells may be obtained from normal or diseased tissue. In some embodiments, cells are obtained from a donor, and their state or type is modified ex vivo using a method of the invention. In certain embodiments of the invention a cell may be a free cell in culture, a free cell obtained from a subject, a cell obtained in a solid biopsy from a subject, organ, or solid culture, etc.

A population or plurality of isolated cells in any embodiment of the invention may be composed mainly or essentially entirely of a particular cell type or of cells in a particular state. In some embodiments, an isolated population of cells consists of at least 30%, 40%, 50%, 60%, 70%, 80%, 90%, 95%, 96%, 97%, 98%, 99%, or 100% cells of a particular type or state (i.e., the population is at least 30%, 40%, 50%, 60%, 70%, 80%, 90%, 95%, 96%, 97%, 98%, 99%, or 100% pure), e.g., as determined by expression of one or more markers or any other suitable method.

### Controls

Certain embodiments of methods of the invention used to assess one or more of: an effect of an ecDNA on a target oncogene, to the status of a cell with respect to ecDNA/oncogene interactions, an effect of a candidate therapeutic on an interaction between an ecDNA and its target oncogene, etc. Such assessments of ecDNA/target oncogene characteristics in cells, tissue, and/or subjects may be made by comparing results obtained in a sample cell, tissue, or subject with results obtained in a control cell tissue, or subject respectively. As a non-limiting example, some embodiments of the invention include determining the status of one or more ecDNA target oncogenes in a sample cancer cell and in a control cancer cell and comparing the results as a measure of the difference in status of the sample cancer cell and the control cancer cell. In another non-limiting example, a status of an ecDNA/target oncogene interaction is identified in a subject having a cancer, the subject is subsequently administered a candidate therapeutic agent intended to disrupt the identified ecDNA/target oncogene interaction and the status compared before and after administration of the candidate therapeutic agent. It will be understood that results obtained from the subject not yet contacted with the candidate therapeutic agent may be referred to as “control results” and the non-contacted subject as “a control subject”.

As used herein a control may be as described above and also may be a predetermined value, which can take a variety of forms. It can be a single cut-off value, such as a median or mean. It can be established based upon comparative groups. Other examples of comparative groups may include cells or subjects that have a specific cancer or ecDNA/target oncogene status and cells or subjects without the specific cancer or ecDNA/target oncogene status. Another comparative group may be a subject from a group with a family history of a cancer and a subject from a group without such a family history. A predetermined value can be arranged, for example, where a tested population is divided equally (or unequally) into groups based on results of testing. Those skilled in the art are able to select appropriate control groups and values for use in comparative methods of the invention.

Candidate therapeutic agent identification methods of the invention may be carried out in a cell or cells that are in a subject or in cultured or in vitro host cells. Candidate-therapeutic agent identification methods of the invention that are performed in a subject, may include delivery of a candidate agent that is intended to disrupt an ecDNA/target oncogene interaction into a cell in the subject, and assessing the ecDNA/target oncogene interactions and oncogene status (before and/or after delivering the candidate therapeutic agent. A result of contacting a host cell, tissue, and/or subject with a candidate therapeutic agent can be measured and compared to a control value as a determination of an efficacy of the candidate therapeutic in disrupting an ecDNA/target oncogene interaction.

### Compositions

A composition used in a method of the invention can but need not be a pharmaceutical composition. The term “pharmaceutical composition” as used herein, means a composition that comprises at least one pharmaceutically acceptable carrier that is useful in preparing a pharmaceutical composition that is generally safe, non-toxic, and neither biologically nor otherwise undesirable. A pharmaceutical composition may be used in certain embodiments of methods of the invention, a non-limiting example of which is for administering a candidate therapeutic agent to a cell or subject to disrupt an ecDNA/target oncogene interaction.

In certain aspects of the invention a pharmaceutical composition comprises one or more therapeutic or candidate therapeutic agents, with one or more additional molecules, therapeutic agents, candidate agents, candidate treatments, and therapeutic regimens that are also administered to the cell and/or subject. A pharmaceutical composition used in an embodiment of a method of the invention may include an effective amount of a candidate therapeutic agent to do one or more of: reducing an ecDNA/target oncogene interaction, alter the status of a target oncogene transcription in a cancer cell, etc. In some embodiments of the invention, a pharmaceutical composition of the invention may include a pharmaceutically acceptable carrier.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. In certain embodiments of the invention, such preparations may contain salt, buffering agents, preservatives, compatible carriers, aqueous solutions, water, etc.

Delivery of a therapeutic agent to a cell or a subject may be achieved by various means described herein and other art-known means. Such administration may be done once, or a plurality of times. If administered multiple times to a subject, one or more therapeutic agents may be administered via a single or by different routes. For example, though not intended to be limiting: a first (or the first few) administrations may be made directly into a tissue in the subject to be treated, and later administrations may be systemic.

The amount of a therapeutic agent delivered to a cell or subject may, in certain embodiments of the invention, be an amount that statistically significantly reduces an interaction of an ecDNA and its target oncogene. Suitable amounts can be readily determined by a practitioner using teaching provided herein in conjunction with art-known methods, for example clinical trials, and without a need for undue experimentation.

### EXAMPLES

### Example 1

Inside the nucleus, chromosomes are extensively folded into chromatin loops which occupy distinct chromatin territories [Zheng, S. et al. (2013) Genes Dev 27, 1462-1472]. Such highly organized 3-dimensional chromatin conformation provides a topological basis for many genome functions, including transcription, by bringing distal regulatory elements and their targeted genes into close spatial proximity [Cremer, T. & Cremer, M. (2010) Cold Spring Harb Perspect Biol 2, a003889]. Alteration of chromatin conformation as a result of chromosomal rearrangements has been implicated in many human diseases, particularly in cancers [Sexton, T. & Cavalli, G. (2015) Cell 160, 1049-1059]. To understand chromatin organization of ecDNA, and how this contributes to gene transcription regulation, chromatin interaction assessments methods, such as ChIA-PET [Taberlay, P. C. et al. (2016) Genome Res 26, 719-731] methods were applied. Methods were designed that integrated both general spatial chromatin organization [Zhang, Y. et al. (2013) Nature 504, 306-310] and protein factor mediated long-range chromatin interactions, on the same neurosphere cell lines. Studies demonstrated that known ecDNAs were readily recognizable through their intense and aberrant intra- and inter-molecular genome-wide chromatin contacts. In addition, in deciphering the RNA polymerase II (RNAPII)-mediated ecDNA connectomes and their chromosomal partners, an association between ecDNA and actively expressed autosomal oncogenes was identified. This relationship supports a finding that ecDNA function as mobile transcriptional enhancers to promote tumor progression.

### Methods

### Cultures of GBM Patient Tumor-Derived Neurosphere Cells

Neurosphere cell lines were generated and cultured as described [deCarvalho, A. C. et al. (2018) Nat Genet 50, 708-717]. Brain tumor specimens were obtained with written informed consent from patients with protocol approved by the Henry Ford Hospital Institutional Review Board. Briefly, tumor specimens were dissociated and cultured as neurospheres in DMEM/F12 medium (11330-032, Gibco) supplemented with N-2 supplement (17502-048, Gibco) and growth factors (EGF and FGF-basic). Cells with passages between 15 and 26 were collected for experiments.

### ChIA-PET Experiments and Data Analysis

Ten million cells were dual-crosslinked with 1.5 mM EGS (21565, Thermo Fisher) for 45 min followed by 1% formaldehyde (F8775, Sigma) for 20 min at room temperature (RT) and then quenched with 0.125 M Glycine (G8898, Sigma) for 10 min. The crosslinked cells were washed with 1× PBS twice and lysed in 100 μL of 0.55% SDS with incubation at room temperature, 62° C. and 37° C. sequentially for 10 min each, which was followed by 37° C. for 30 min with addition of 25 μL 25% Triton-X 100 to quench the SDS and 37° C. overnight with addition of 50 μL AluI (R0137L, NEB), 50 μL 10× CutSmart buffer and 275 μL H₂O to fragmentize the chromatin. The pelleted digested nuclei were resuspended in 500 μL of dA-tailing solution containing 50 μL 10× CutSmart buffer, 10 μL BSA (B9000S, NEB), 10 μL of 10 mM dATP (N04405, NEB), 10 μL Klenow (3′-5′ exo-) (M0202L, NEB), and 420 μL H₂O with 1 h incubation at RT and then subjected to proximity ligation by adding 200 μL 5× ligation buffer (B6058S, NEB), 6 μL biotinylated bridge linker (200 ng/μL), 10 μL T4 DNA ligase (M0202L, NEB) and 284 μL H₂O and incubating at 16° C. overnight. The ligated chromatins were then sheared by sonication and immunoprecipitated with anti-RNAPII antibody (920102, Biolegend). The immunoprecipitated DNA tagmentation, biotin selection, library preparation and sequencing were performed as described [Tang, Z. et al. (2015) Cell 163, 1611-1627]. ChIA-PET Utilities, a scalable re-implementation of ChIA-PET Tools [Li, G. et al. (2010) Genome Biol 11, R22] (see code availability), were used to process ChIA-PET data. After removing the sequencing adaptors, the paired-end reads with a bridge linker were identified and the tags flanking the linker were extracted. Tags identified (≥16 bp) were mapped to hg19 using BWA alignment [Li, H. & Durbin, R. (2009) Bioinformatics 25, 1754-1760] and mem [Li, H. (2013) arXiv:1303.3997 [q-bio.GN]1 according to their tag length. The uniquely mapped, non-redundant pair-end tags (PETs) were classified as inter-chromosomal (Left tags and Right tags from different chromosomes), intra-chromosomal (Left tags and Right tags with genomic span ≥8 Kb) and self-ligation PETs (Left tags and Right tags with genomic span <8 Kb). Both the inter- and intra-chromosomal PETs were extended by 500 bp. PETs overlapping at both ends were then clustered as iPET-2, 3 . . . . Interactions that overlapped with chr M, chr Y were not examined in this study. To remove the potential noise that resulted from genome sequence context and tagmentation bias created by the Tn5 digestion, the interactions in which anchors overlapped with the blacklist (see below on how it was defined) were filtered. For intra-chromosomal interactions, the statistical assessments of interaction significance were performed using ChiaSigScaled, a scalable re-implementation of ChiaSig [Paulsen, J. et al. (2014) Nucleic Acids Res 42, e143]. Significant interactions defined as iPET≥3, FDR<0.05 and inter-chromosomal interactions as iPET≥2 were used in all downstream analysis except for the nsTIF analysis, which used all reported inter-chromosomal interactions (see section below on the discovery of known ecDNA regions from in situ ChIA-PET data). RNAPII binding peaks were called with all uniquely mapped reads using MACS2 (options: --keep-dup all --nomodel --extsize 250) [Liu, T. (2014) Methods Mol Biol 1150, 81-95]. To define intra-ecDNA interactions, ecDNA regions were used to collect all interactions within the reported regions. For ecDNA mediated trans-chromosomal interactions, only interactions that originated from chromosomes outside the chromosomes where ecDNA resided were included. The RNAPII binding status at both anchors of the interactions were checked and RNAPII mediated interactions defined as the interactions with RNAPII binding at both anchors. The interactions were further classified based on their anchors overlapped with GENCODE gene models (Release 19, excludes all pseudogene and all RNAs except miRNA) with priority given to promoter (P) region (defined as ±2.5 kb of TSS) followed by gene region (G). Anchors that do not overlap with any intragenic region were classified as intergenic (I). The oncogenes from the union list of NCG 6 [Repana, D. et al. (2019) Genome Biol 20, 1] and COSMIC v87 [Forbes, S. A. et al. (2015) Nucleic Acids Res 43, D805-811] were used to annotate the ecDNA-interacting genes.

### Blacklist Regions

To remove biases introduced by ChIA-PET experimental procedures, such as over tagmentation by Tn5 in certain loci on the genome, a greylist was created using 8 human cell ChIA-PET libraries made from different antibody enrichment (4 with anti-CTCF antibody and 4 with anti-RNAPII antibody), down-sampled to represent equal number of reads totaling 75,215,727 tags. Peaks were called from the merged data set using MACS2.1.0.20151222 [Liu, T. (2014) Methods Mol Biol 1150, 81-95] with FDR<0.05, which resulted in 153,735 peaks. These peak regions on autosomal and X chromosomes were candidates for the greylist. The regions with shorter peaks (which likely is due to the Tn5 tagmentation) were further filtered while keeping the highest confidence measured by the q values and pileup by the following criteria: region length <600 bp, top 1% highest pileup, 10% lowest q values, and top 10% highest in fold-enrichment of the peaks. These 3 quantities or vectors (inverse length, fold-enrichment, and pileup) of the filtered peaks (1,119 regions with q values <1E-165 and fold-enrichment from 10 to 50) were taken and each of the vectors (with R command ‘scale (center=F, scale=T)’) were scaled. Next, the three vectors were individually normalized so that the average of each vector was 1. A scoring function was defined, s=fold-enrichment+pileup+1/length. The greylist consisted of the 321 peak regions with score s above the average. Additionally, four regions were included that appeared to be artifacts from visual inspection. The final blacklist that was adopted was a concatenation of the ChIA-PET greylist and the publicly available blacklist from Kundaje lab (github.com/kundajelab/HiC-pipeline/blob/master/hic_flexibleWindow-pipeline/data/reference genomes/hg19/wgEncodeHg19ConsensusSignalArtifactRegions.bed.gz).

Discovery of Known ecDNA Regions From In-Situ ChIA-PET Data

The processed interaction data from RNAPII ChIA-PET from each cell line was aggregated into a genome-wide interaction frequency (IF) matrix M_{N×N}={Mᵢⱼ|i,j=1, 2, . . . , N}. The hg19 genome was segmented at 50 Kb intervals into 60,739 non-overlapping bins from the start of chromosome (the last bin of each chromosome might not represent full 50 Kb). Bins that overlapped with blacklist (see below) were removed from the IF matrix. Known ecDNA regions exhibited large numbers of interactions both within the ecDNA regions and widespread throughout all 23 chromosomes, yielding very high IF sums, particularly between different chromosomal regions amplified on ecDNAs. Methods used took advantage of these features to test whether known ecDNA regions [deCarvalho, A. C. et al. (2018) Nat Genet 50, 708-717] could be uncovered from the ChIA-PET interaction data, and possibly prediction of additional genomic regions amplified on ecDNAs.

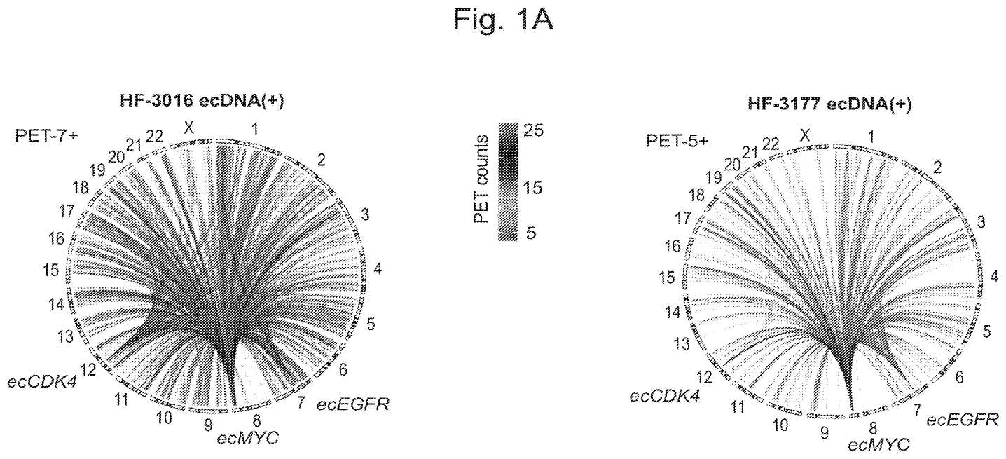

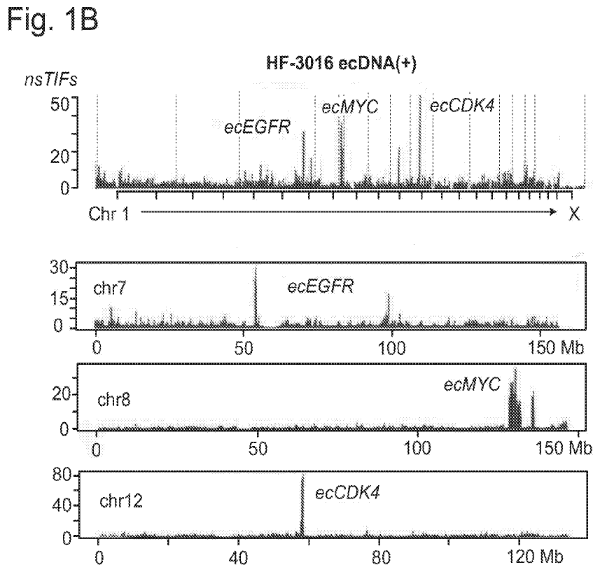

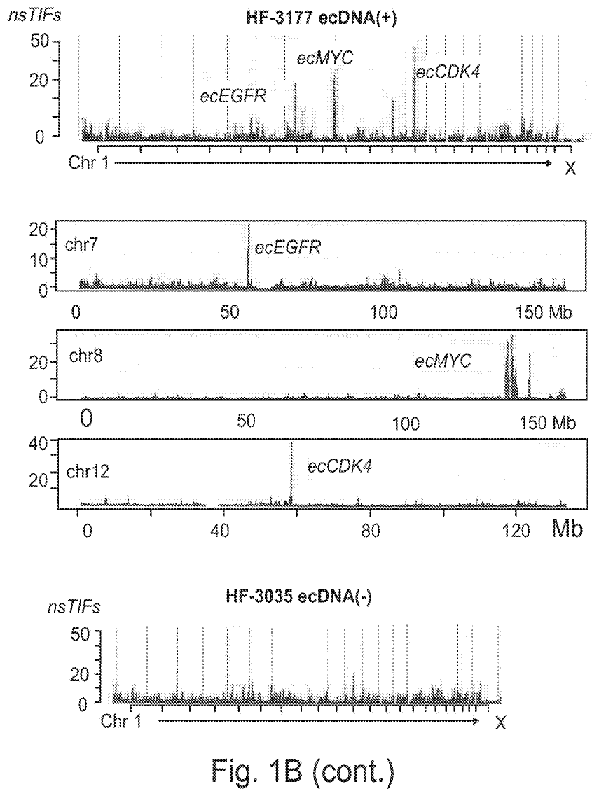

The sums of trans-chromosomal IF (TIF) for every bin were computed and normalized so that they are comparable across different libraries. This data vector was scaled (divided by its magnitude and multiplied by the length of vector) so that the mean was equal to 1. Let the i-th bin of this normalized vector be nsTIFᵢ. The bins with highest nsTIF were compared with the known ecDNA regions in ecDNA (+) data. To understand the distribution of nsTIF in ChIA-PET data from ecDNA (−) cells, genome-wide distribution of nsTIF was examined in HF-3035 cells (FIG. 1B) as well as other pluripotent cell lines (data not shown) and found them to be all less than 20. Therefore, a threshold was introduced as the first pass to determine the ecDNA candidate regions. An additional threshold for nsTIF was also introduced to prioritize bins as candidates for ecDNA. Based on the knowledge, genomic regions amplified by ecDNAs did not exceed more than 0.1% of the genome size, so the low-threshold (t₁) was set as the mean of nsTIF, in the top 0.1%. Empirically, the high-threshold (tₕ) was set to 25 (i.e. 25 times higher than expected) if t₁<25 otherwise tₕ=max(nsTIF). All i-th bins with nsTIFᵢ>tₗ were put in a list of candidates. On the other hand, no region would be considered as ecDNA if all nsTIFᵢ<tₕ. In this study, the highest nsTIF from ecDNA (−) data was lower than 21, which was substantially lower than that in ecDNA (+) data sets (˜38 to 82).

The list of candidates was grouped based on their genomic distance, such that groups located near each other were grouped together, i.e. the minimum distance between 2 groups was 1 Mb. The purpose of grouping was to score every candidate according to committed connections (measured by IF) to candidates in different groups. The score was based on how many groups every candidate was connected to. A ‘connection’ was defined if the candidate's normalized TIF was higher than a threshold, t_{T}. Let the normalized interchromosomal element of the matrix M be Tᵢⱼ and the normalized TIF vector for bin i was Tᵢ (the same normalization as nsTIF, i.e. Tᵢⱼ={Mᵢⱼ/Σⱼ^{Ntran}Mᵢⱼ*Ntran, where j are trans-chromosomal to index i}). Then a connection was present if their interaction frequency was relatively high, e.g. there was at least one bin, e.g., k-th bin, in another group of candidates with Tᵢₖ>_{T}, where t_{T} is the higher value of 100 and the average of top 0.1% Tᵢ. The connection score +1 was then added to candidate i.

The group of candidates with the highest nsTIF was then checked first to see if all of their connection scores were zero. When this condition was true, it was proposed that all of the candidate regions in this single group were ecDNA regions if there were no more than 5 groups with nsTIF≥tₕ, otherwise no ecDNA regions were predicted (noisy data might show many groups exhibit nsTIF above the high-threshold with 0 connectivity score which were likely false positive). When the condition was false, i.e., there were multiple groups with nonzero connection scores, it was predicted that regions from those groups with connection score greater than zero are ecDNA. Additional studies are performed to assign the specificity of the regions amplified on ecDNAs from ChIA-PET data.

### ChIP-Seq Library Construction and Data Analysis

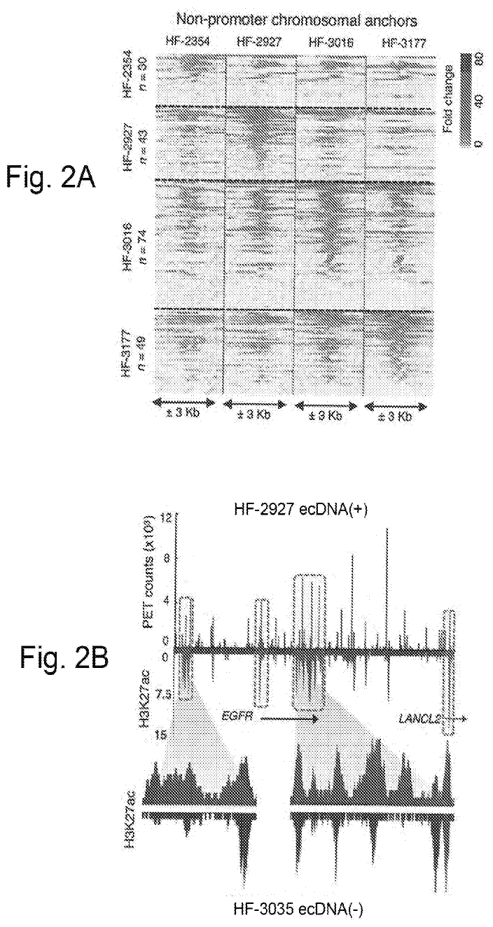

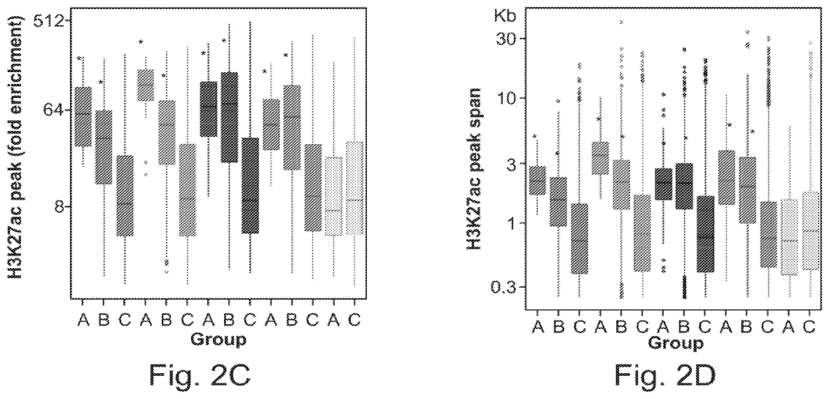

Two million cells were crosslinked and lysed in the same way as ChIA-PET. After lysis, the nuclei pellets were sonicated and immunoprecipitated with anti-H3K27ac antibody (39133, Active Motif). 4 ng DNA from both antibody immunoprecipitation and input were subjected to end-repair, A-tailing and adaptor ligation with the KAPA Hyper Prep Kit (KK8505, KAPA Biosystems). Adaptor-ligated DNA fragments were PCR amplified with the KAPA Library Amplification ReadyMix (KK2612, Kapa Biosystems) and sequenced on Illumina platform with 75 bp single-end sequencing. The raw reads were quality trimmed using Trim Galore version 0.4.3 (options: --stringency 3 -q 30 -e 0.20 --length 15) and mapped to the hg19 genome using BWA 0.7.12 (command: aln) [Li, H. & Durbin, R. (2009) Bioinformatics 25, 1754-1760]. The uniquely mapped and de-duplicated reads were used for peak calling (FDR<0.05) with MACS2.1.0.20151222 (options: --nomodel --extsize 250 -B --SPMR -g hs) [Liu, T. (2014) Methods Mol Biol 1150, 81-95]. Peaks defined with FDR<0.05 were used in all analyses. For H3K27ac analysis in FIG. 2C-D, additionally requirement for P<0.001 was applied.

### Immuno-Staining

The unfixed metaphase cells were dropped to the slides and preincubated in KCM buffer (120 mM KCl, 20 mM NaCl, 10 mM Tris-HCl, pH 8.0, 0.5 mM EDTA, 0.1% (v/v) Triton X-100) for 10 min at RT. Slides were blocked in 1% (w/v) BSA/KCM buffer for 30 min at RT, followed by incubation with primary H3K27ac antibody (39133, Active Motif) in 2% BSA overnight at 4° C., KCM buffer for 10 min at RT twice, goat anti-rabbit Alexa Fluor 488 secondary antibody (A32731, Invitrogen) for 30 min at RT. After washing with KCM buffer twice, slides were crosslinked with in 4%(v/v) formaldehyde/KCM for 15 min, mounted the coverslip with 50 μL Prolong Gold Antifade (Invitrogen) and sealed with clear nail polish. The slides were scanned under Leica STED 3×/DLS Confocal.

### Transcription Factor Motif Analysis

Homer2 [Heinz, S. et al. (2010) Mol Cell 38, 576-589] searched against 414 known motifs in 206 target sequences against a normalized background of 71,232 H3K27ac peak regions. The search results are selected based on the criteria: q value<0.001, enrichment>1.5, % of target sequences with motif>25%.

### RNA-Seq Library Construction and Data Analysis

Total RNA was isolated in biological replicates using AllPrep DNA/RNA Mini Kit (80204, QIAGEN). Strand-specific RNA libraries were generated from 300 ng of total RNA using KAPA Stranded mRNA Sequencing Kit (KK8502, KAPA Biosystems) following the manufacturer's instruction. Libraries were sequenced on the Illumina platforms with 75 bp paired-end sequencing. The raw sequencing reads were trimmed using Trim Galore version 0.4.3 (options: --stringency 3 -q 20 -e .20 --length 15 --paired) and aligned to the hg19 genome using hisat 2.1.0 (options: --dta-cufflinks). The transcripts were assembled using Cufflinks 2.2.1 [Trapnell, C. et al. (2010) Nat biotech 28, 511-515] and the final expression level were quantified using Cuffdiff (options: --library-type fr-firststrand) [Trapnell, C. et al. (2013) Nat biotech 31, 46-53]. The correlation between the sequencing data from different samples was analyzed with R/pheatmap package (version 1.0.2.).

Define ecDNA-Mediated Chromatin Interaction Community

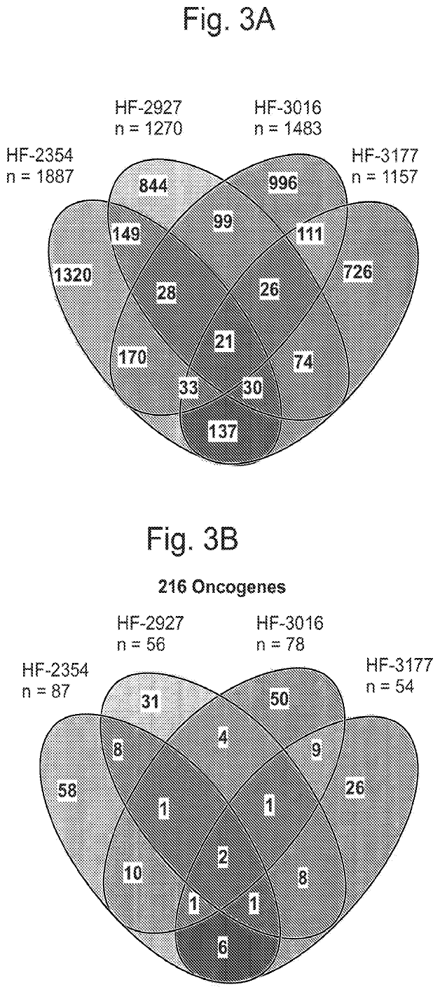

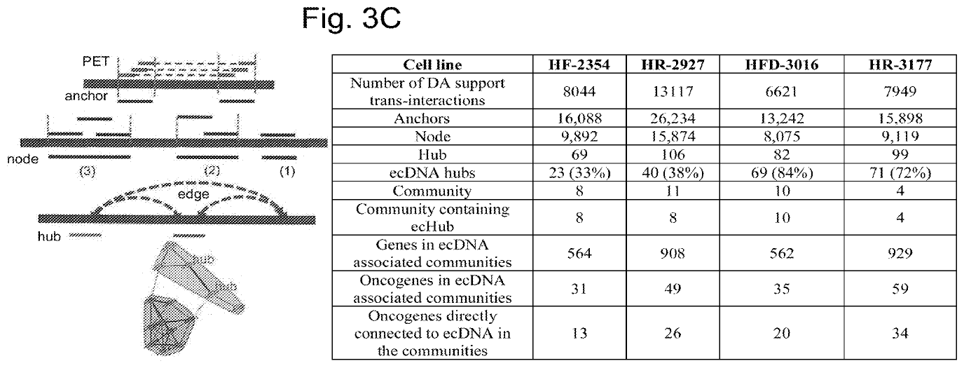

Collection was done of all trans-chromosomal interactions that are supported by RNAPII binding on both anchors. All RNAPII bound anchors from HF-2354, HF-2927, HF-3016 and HF-3177 cell lines were pooled and anchors overlapped with blacklist regions were removed to merge into 29,721 non-overlapping interaction nodes (FIG. 3C). For each node, a connectivity score was defined, i.e., the number of interaction partner nodes it was connected to. Hubs were defined as nodes with connectivity scores higher than 3 times standard deviation above average. With this parameter, hubs have >10 connection links to other nodes. In total, 69, 106, 82, and 99 hubs were defined from HF-2354, HF-2927, HF-3016 and HF-3177 cell lines, respectively. Using hubs-hubs networks, communities were generated with cluster_edge_betweenness function in igraph library in R [Csardi, G. & Nepusz, T. (2006) Computer Science]. The number of ecDNA associated communities are 8, 8, 10, and 4 from HF-2354, HF-2927, HF-3016 and HF-3177 cell lines, respectively.

### Data Availability Statement

All data described in this study are being deposited in NCBI's Gene Expression Omnibus GSE124769 with a reviewer linker key ‘uxkhaeyctdixts’ using the following link www.ncbi.nlm.nih.gov/geo/query/acc.cgi?acc=GSE124769.

### Code Availability

Code for ecDNA detection from ChIA-PET data is available at www. dropbox. com/sh/2crbfj1kr2yyws/AACLiUg6Ch9y6FurbbUzmcbPa?dl=0. ChIA-PET Tools (code available at github.com/cheehongsg/CPU) ChiaSigScaled (code available at github.com/cheehongsg/ChiaSigScaled)

### Results and Discussion

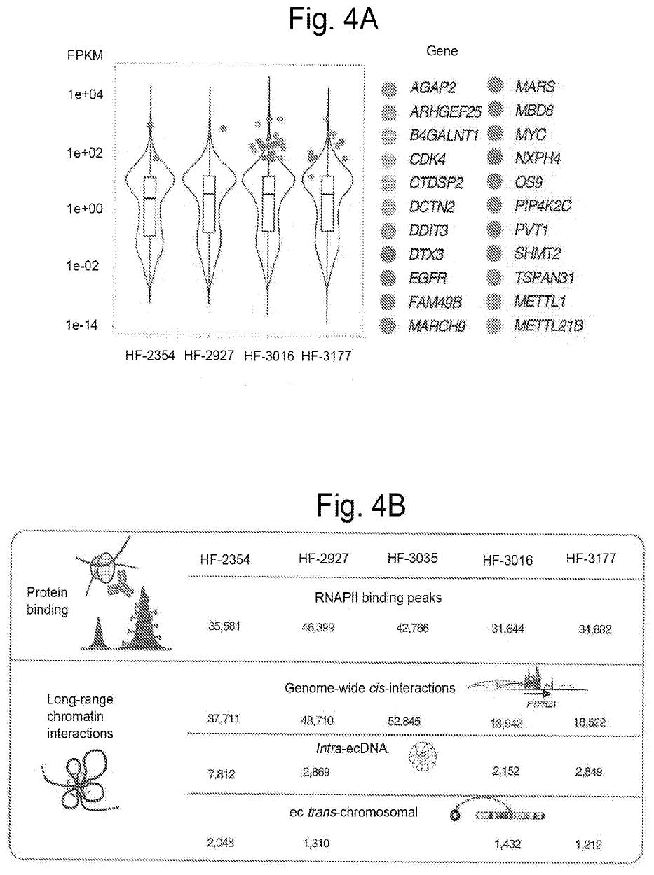

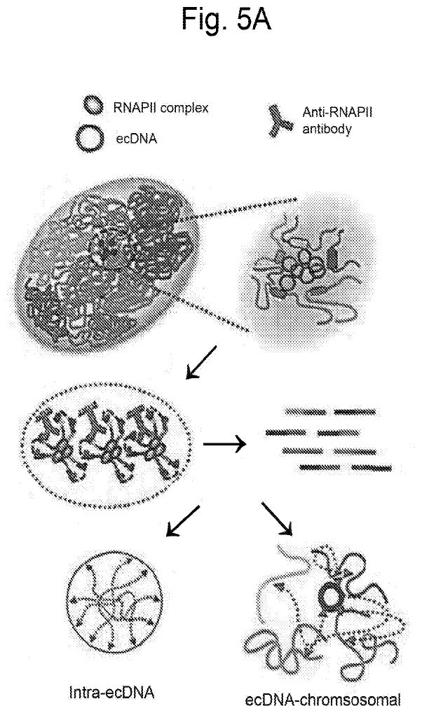

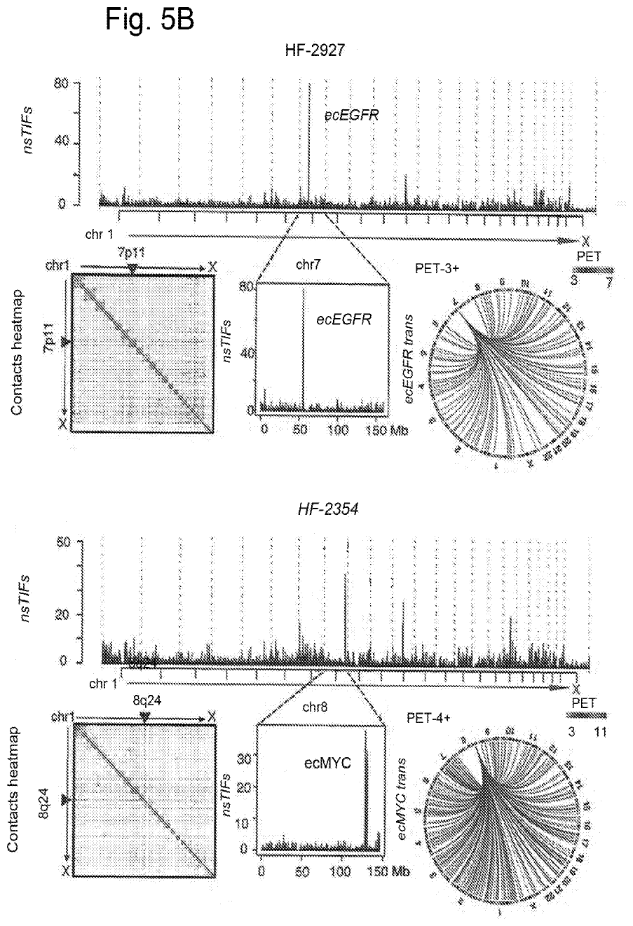

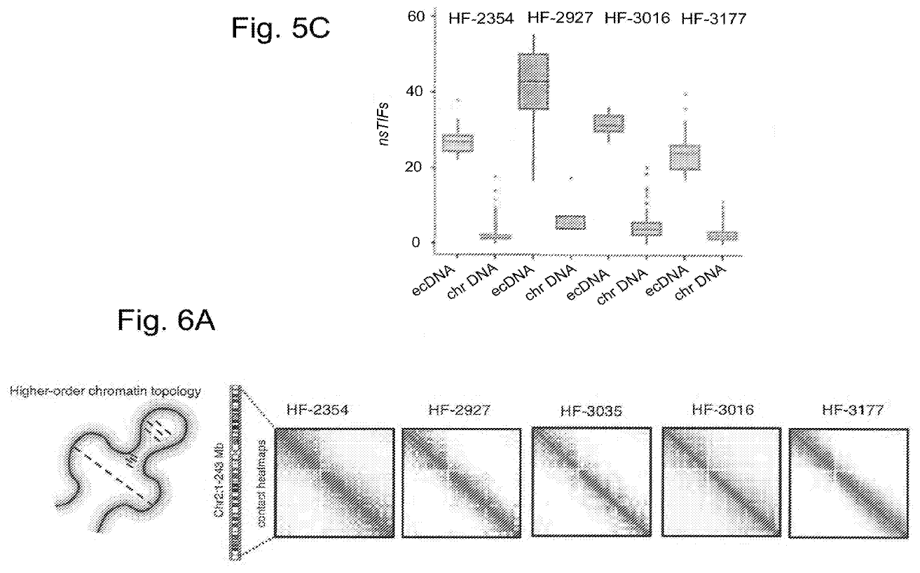

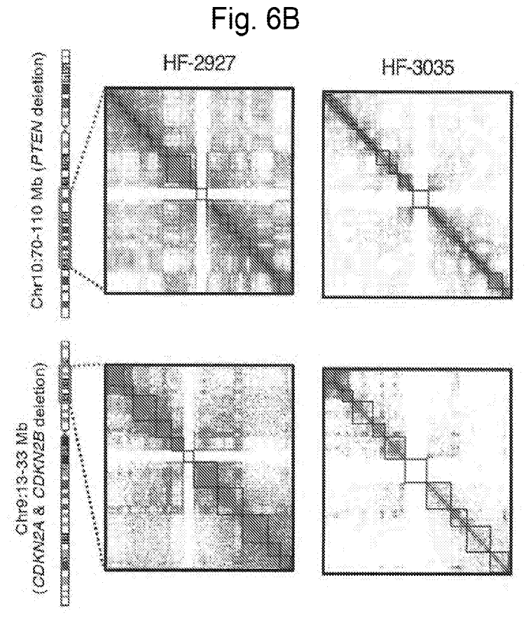

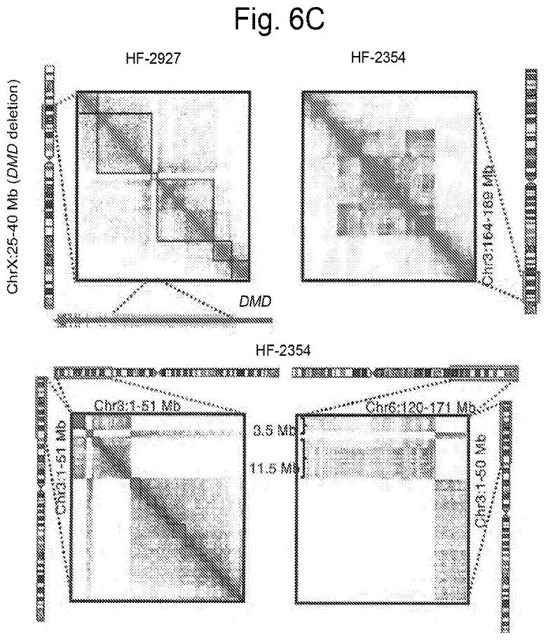

ChIA-PET analysis was performed on five GBM patient-derived neurosphere cell lines whose ecDNA status were previously established from whole genome sequencing data and confirmed by fluorescence in-situ hybridization (FISH) analysis. Four of the five neurosphere lines are ecDNA (+) (HF-2354, HF-2927, HF-3016 and HF-3177) and one line is ecDNA (−) (HF-3035) [deCarvalho, A. C. et al. (2018) Nat Genet 50, 708-717]. Based on the high level of RNA expressed from genes amplified within the ecDNAs (FIG. 4A), it was reasoned that ecDNA was highly associated with RNAPII within the active chromatin domains. RNAPII chromatin immunoprecipitation was used to pull down RNAPII-associated chromatin and used the ChIA-PET assays to characterize ecDNA-chromatin interactomes (FIG. 5A). The resulting ChIA-PET data detected both RNAPII binding sites, long-range chromatin interactions between regulatory elements [Zhang, Y. et al. (2013) Nature 504, 306-310] (FIG. 4B) as well as non-enriched chromatin contacts within spatial topologically chromatin associated domains (TADs) [Dixon, J. R. et al. (2012) Nature 485, 376-380] (FIG. 6A). Chromosomal structural variants, such as deletions of PTEN and CDKN2A & CDKN2B on chromosomes 10q23 and 9p21, resulted in an elimination of chromatin contacts (FIG. 6B). Other examples included the detection of a 600 Kb deletion of the chrX:31.4-32 Mb common fragile site [Ma, K. et al. (2012) Int J Mol Sci 13, 11974-11999] involving the DMD gene in HF-2927, a 15 Mb extensive rearrangement of chr3:168-183 Mb as well as a double translocation event of 3.5 and 11.5 Mb between chr3 and chr6 in HF-2354 genomes (FIG. 6C).

HF-2927 harbors a chr7p111EGFR containing ecDNA [deCarvalho, A. C. et al. (2018) Nat Genet 50, 708-717], referred to as ecEGFR, whereas HF-2354 contains a chr8q24/MYC ecDNA, referred to as ecMYC. In HF-3016 and HF-3177, two neurosphere lines derived from a primary and a recurrent GBM from the same patient, three genes were found to be amplified extrachromosomally [deCarvalho, A. C. et al. (2018) Nat Genet 50, 708-717], of which chr7p111EGFR and ch12q14.1/CDK4 were demonstrated to be co-amplified on ecDNA while chr8q24/MYC was also found on ecDNA (referred to as respectively ecEGFR, ecCDK4 and ecMYC). All ecDNA loci exhibited extensive contacts throughout the entire genome (FIG. 5B, FIG. 1A), suggesting the high trans-connectivity of these ecDNA regions to the regions across all chromosomes. To quantify the degree of trans-chromatin contacts, a metric was developed that faithfully describes the genome-wide trans-interaction frequencies (nsTIF) normalized across all 23 chromosomes and applied this metric on each of the five neurosphere cell lines (see Methods herein). EcDNA regions showed highly elevated nsTIF levels (maximal nsTIFs were ˜38-82) in all four ecDNA (+) lines but not in the HF-3035 ecDNA (−) cells (nsTIFs<21) (FIG. 5B, FIG. 1B). The nsTIF spike regions matched closely with the ecDNA regions. Furthermore, the high nsTIF regions linked to ecDNA segments showed trans contacts across the entire genome (FIG. 5B), suggesting a dynamic ecDNA connectivity from the extrachromosomal genetic elements. The characteristic genome-wide contact pattern may be explained by the mobile nature of ecDNA. In the two di-amplicon lines HF-3016 and HF-3177, results showed elevation in ecMYC, ecEGFR and ecCDK4 nsTIF levels (FIG. 1B) as well as cross-interactions between the three loci, suggesting that the dominant ecDNA in these lines carried all three oncogenes or that they have a close inter-molecular proximity. To verify that the high nsTIFs were specific to the extrachromosomal nature of the ecDNAs and not amplification status, nsTIF values from all genomic regions with copy number gain ≥3 (which ranged from 3 to 6) were compared with nsTIFs from the ecDNA regions. The nsTIF values from chromosomally amplified segments, as expected to be constrained within chromosomal territories, were significantly lower (median nsTIFs between 1.5-4 vs. 24-43) than the nsTIFs from ecDNA regions (one-sided Wilcoxon rank-sum test, P value <0.0005) (FIG. 5C), confirming that the elevated contact frequency of ecDNA across the genome is not explained by a DNA dosage effect alone but is determined by its autonomous capacity.

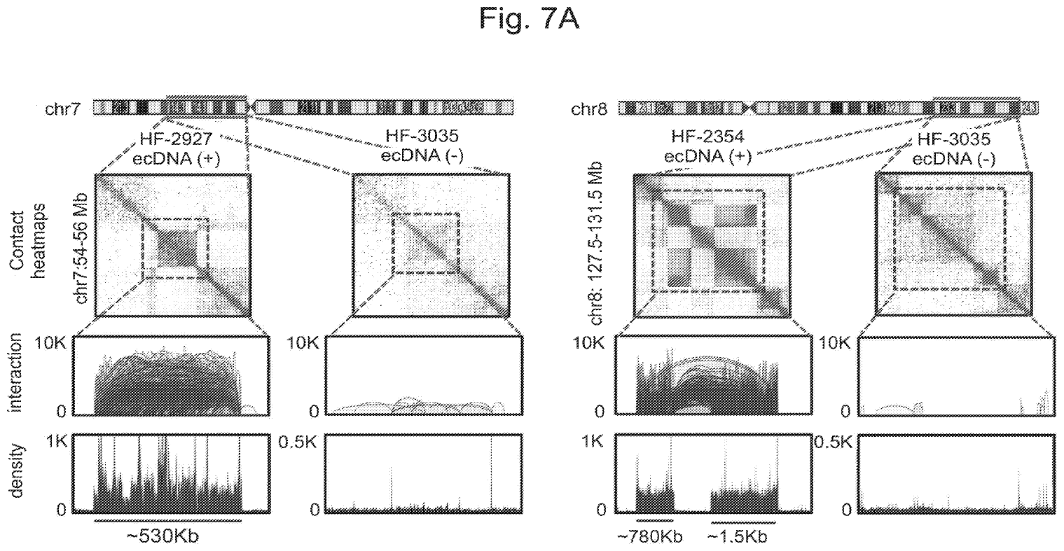

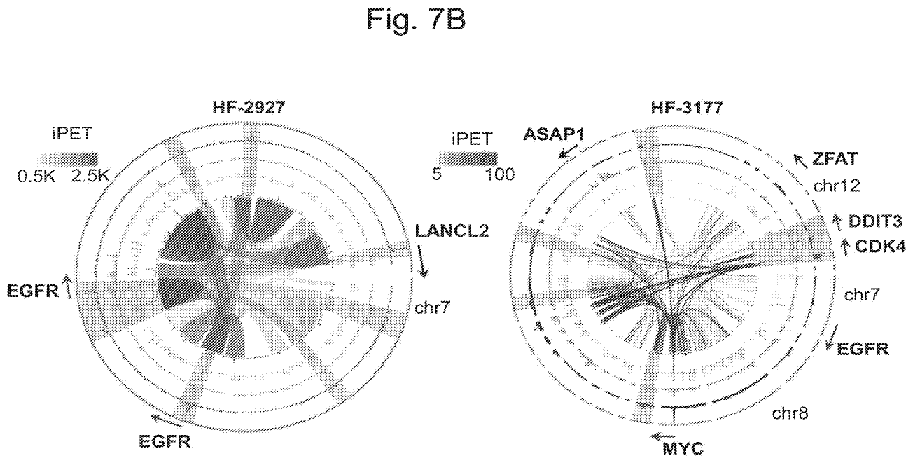

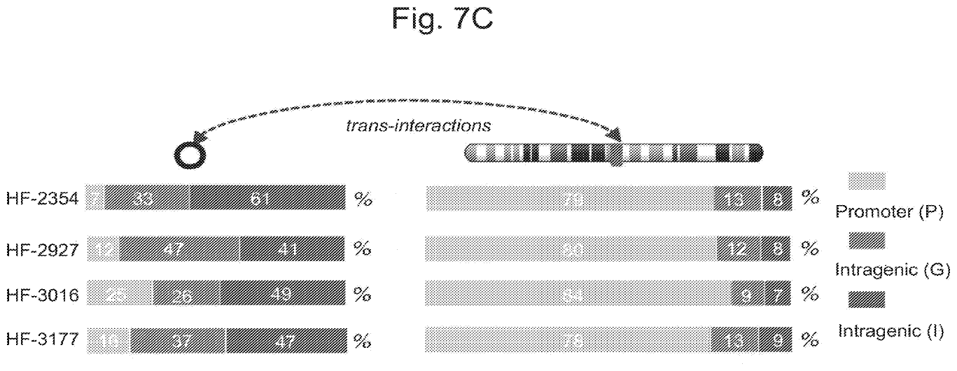

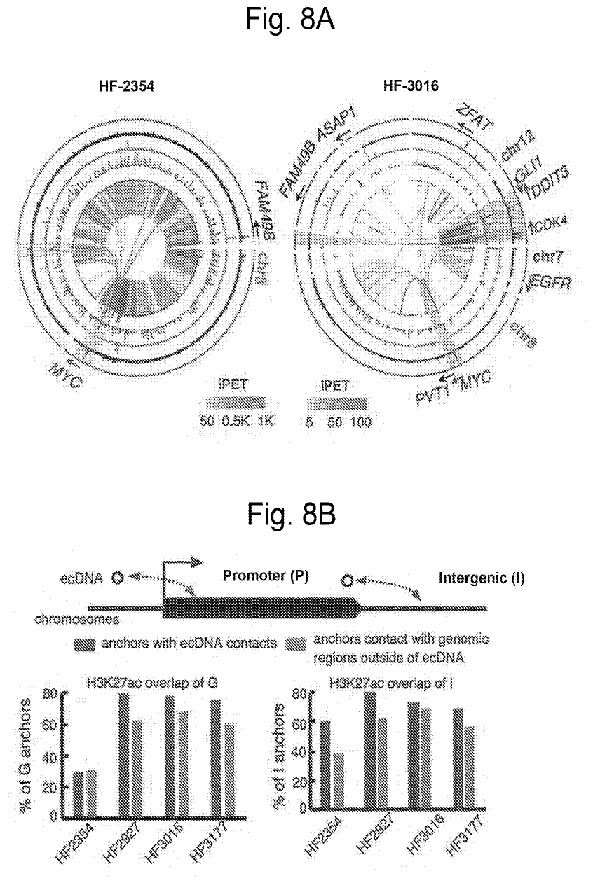

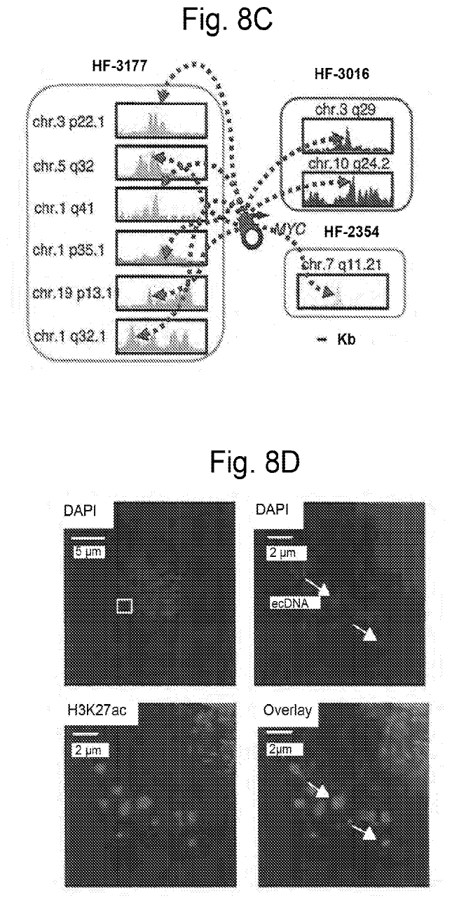

In addition to the high trans-interaction frequency, a strikingly high frequency of cis-interactions was detected within the genomic regions of ecDNAs. In HF-2927 ecDNA (+) cells, the cis-interaction intensity observed within the ˜530 Kb ecDNA region was 2,879, a 240-fold increase, compared to only 12 in the same region in ecDNA (−) HF-3035 cells (FIG. 7A). The high contact increase directly reflected both size and genomic structure of this ecEGFR. Similarly, intensive cis-interactions were observed within and between the two segments of the defined ecMYC region in HF-2354 (FIG. 7A). Extensive RNAPII tethered chromatin contacts (defined as RNAPII binding detected at DNA regions connected by the interactions, referred as anchors) were detected both in cis between different regions within ecDNA (referred as intra-ecDNA) (FIG. 7B, FIG. 8A) and in trans (referred as trans-interactions) with other genes or regulatory elements on linear chromosomes (FIG. 7C). The intra-extrachromosomal connectivity patterns showed distinct pairs of loops with high frequency interactions and foci of intense contacts (FIG. 7B, FIG. 8A), which may collectively derive from contacts between different ecDNA molecules and folding within individual ecDNAs. This hypothesis is corroborated by the primary and matching recurrent neurosphere lines (HF-3016 vs. HF-3177), while both lines contain ecMYC, ecEGFR and ecCDK4, 92% and 95% of the intra-ecDNA loops detected in HF-3016 and HF-3177 are exclusively found in their respective cells. Intra-ecDNA loops detected in HF-3016 are predominantly in the ecCDK4 locus (inner circles in FIG. 8A) while in HF-3177, ecMYC regions show more intensive looping (inner circles in FIG. 7B), which could implicate that ecDNAs, although may involve similar oncogene drivers, arise from different and distinct structures. Among the trans-interactions between ecDNAs and their chromosomal partners, the anchors on ecDNAs were mostly (75-93%) in intra- or inter-genic non-coding regions and their trans-interaction chromosomal anchors were mostly (79-84%) localized at promoters (defined as TSS±2.5Kb) (FIG. 7C). Such juxtaposition of the interactions suggests a transcriptional function for these contacts.

To address how ecDNA interactions associate with transcriptional regulatory regions, H3K27ac profiling was performed to mark active enhancers and promoters. First examined was regulation of the oncogenes amplified on ecDNA by evaluating their trans-chromosomal interaction regions. These ecDNA-connecting non-coding chromosomal anchors exhibited high overlap (intergenic 61-80%) with H3K27ac peaks; which was significantly higher than those from the trans-interaction non-coding chromosomal anchors with no ecDNA contact (intergenic 38-69%, FIG. 8B, P value 0.019, one sided Wilcoxon rank-sum test). Specifically, 73% (144 out of 196) of the chromosomal non-coding regions interacting with the promoters of oncogenes residing on ecDNAs overlap with H3K27ac peaks in their corresponding cell lines (FIG. 2A), suggesting that the transcription of the oncogenes on ecDNA is further enhanced by engaging enhancers on the linear chromosomes through chromatin contacts. The enhancer contacts were variable and may be dynamic among different ecDNA (+) lines. In the case of ecMYC, MYC promoter interacted nine different H3K27ac enhancers in three ecMYC (+) cell lines (FIG. 8C).

Co-occurrence was observed between the high frequency contact foci and H3K27ac peaks within the ecDNAs (FIG. 7B, FIG. 8A), suggesting that these interaction anchors behave like active enhancers. The H3K27ac peaks within the 530 Kb ecEGFR region co-aligned with the regions of high interaction frequency in HF-2927, and exhibited the pattern as clusters in close proximity with broader genomic spans when compared to the H3K27ac peaks in the chromosomal EGFR region in the ecDNA (−) cells (HF-3035) (FIG. 2B), indicating enhancer signals accumulated on the chromatin contact sites of ecDNA. Immunostaining using an antibody targeting H3K27ac on metaphase HF2927 cells demonstrated overlapping signals between H3K27ac and DAPI signals marking ecDNA, confirming the association between enhancer function and ecDNA (FIG. 8D).

To quantitatively demonstrate the increases in H3K27ac signal associated with ecDNA mediated trans-chromatin interactions across all four ecDNA (+) cell lines, comparisons were performed of the fold enrichment from all H3K27ac peaks (FDR<0.05, P<0.001) detected between the ecDNA regions (referred to as Group A), their corresponding trans-interacting chromosomal partners (Group B), and the genome-wide H3K27ac peaks which have no contact with ecDNA (Group C) in each of the four ecDNA (+) cell lines. H3K27ac peaks associated with ecDNA chromatin interaction anchors have significantly higher enrichment (median values for Group A:58-138 and Group B:43-91) than those of genome-wide H3K27ac peaks with no ecDNA contacts (median value 10-12, P value 5E-09 to 2.3E-164, one-sided Wilcoxon rank-sum test) (FIG. 2C). They are also higher than the enrichment fold (median value: 9-11) from the ecMYC, ecEGFR and ecCDK4 equivalent regions found in the ecDNA (−) HF-3035 cell line, confirming the strong enhancement of H3K27ac signal is specific to the ecDNAs.

Enhancers with super-high intensity and large domains of H3K27ac signals have been referred to as “super-enhancers” [Whyte, W. A. et al. (2013) Cell 153, 307-319], which have been found to promote oncogenes transcription in cancers [Hnisz, D. et al. (2013) Cell 155, 934-947]. To evaluate whether the enhancer signatures observed in the ecDNA are reminiscent of the “super-enhancers”, examinations were performed of the span sizes of the H3K27ac peaks detected in ecDNAs and their trans-interacting chromosomal anchors. It was found that H3K27ac peaks on ecDNA had significantly longer spans than the chromosomal H3K27 peaks with no ecDNA contact (median spans of 2-3.5 Kb in Group A, 1.5-2.1 Kb in Group B vs. 700-800 bp in Group C, P values 9.6E-08 to 4.4E-153, one-sided Wilcoxon rank-sum test) (FIG. 2D). Sequence analysis of the Group A H3K27ac peaks on ecDNAs in relative to other H3K27ac peak regions showed an enrichment of binding motifs (q value <0.001, enrichment >1.5) of transcription factors critical in regulating RNAPII general transcription and cell proliferation, including JUN, FOS and ATF. Collectively, both cis and trans convergence of RNAPII signal with strong enhancement of H3K27ac signal suggested that ecDNA molecules are able to connect the RNA polymerase machinery broadly across the genome, corroborating a function as a genome-wide transcriptional amplifier.

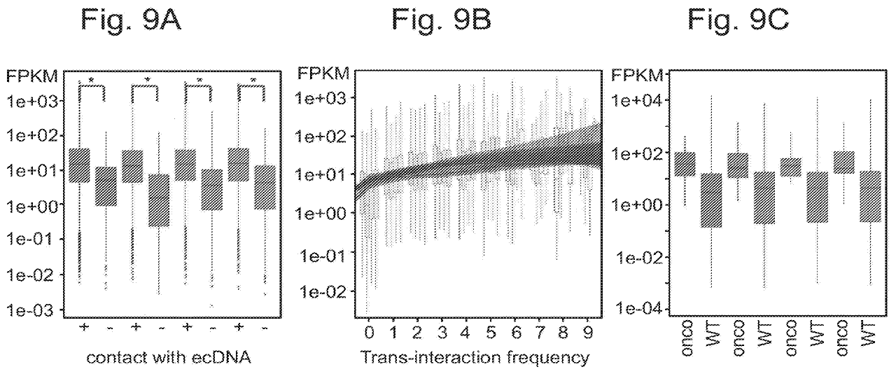

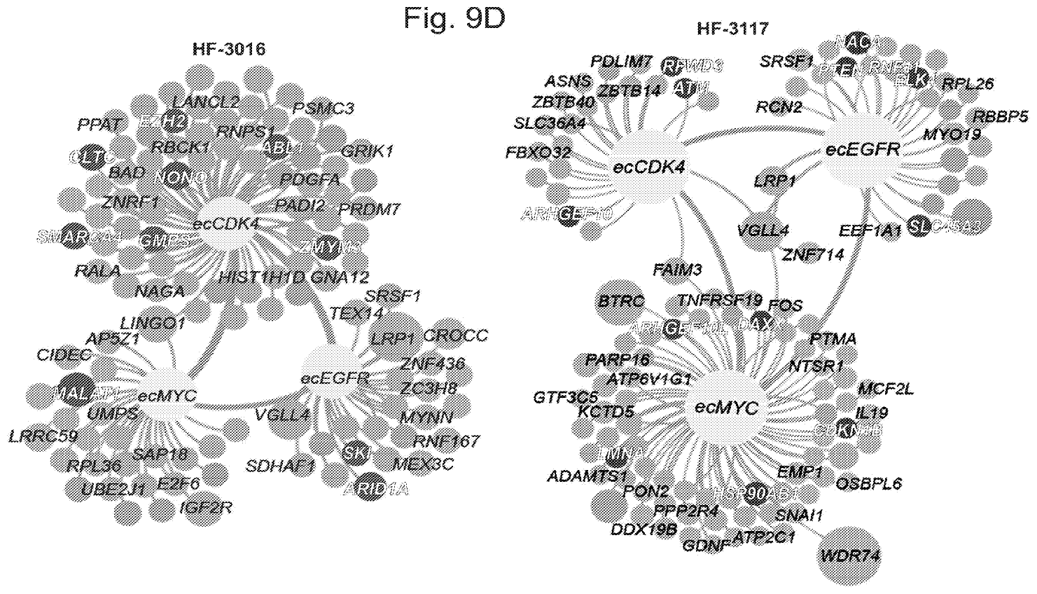

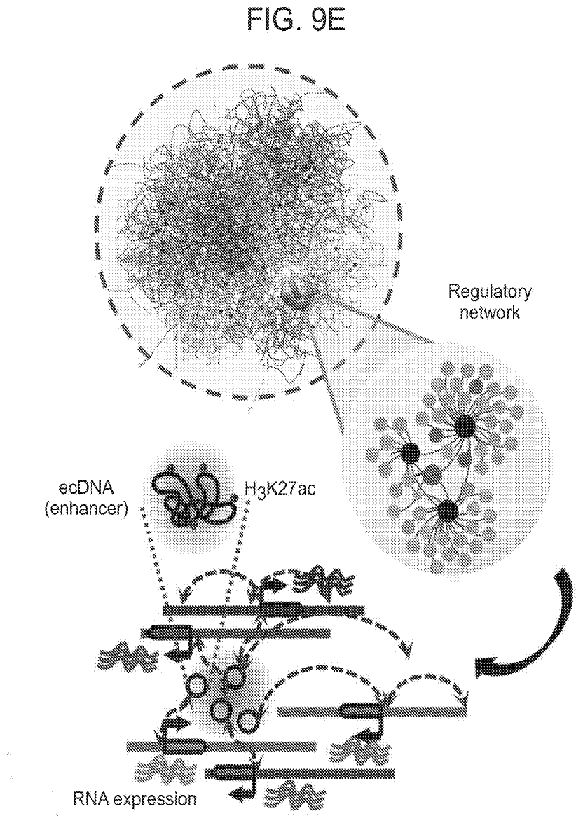

Next, it was determined whether the increase in enhancer signals associated with ecDNA trans-interactions results in active transcription, through the analysis of RNA expression from the same four lines. In total, results indicated detection of 1,887, 1,270, 1,483 and 1,157 chromosomal genes whose promoters made contacts with ecDNAs in HF-2354, HF-2927, HF-3016 and HF-3177 ecDNA (+) cell lines, respectively. EcDNA interacting genes showed significantly higher levels of expression (FPKM median value to 12-14) compared to genes with either no other trans-chromosomal contact (FPKM median value 0.7-4, P value 4.3E-34 to 1.8E-55, one-sided Wilcoxon rank-sum test) (FIG. 9A) or genes with no contact with ecDNAs but having trans-chromosomal interactions with other genes (FPKM median value 8-9, P value 6.1E-04 to 1.5E-08, one-sided Wilcoxon rank-sum test) (FIG. 10). Furthermore, the expression level of the ecDNA-connecting genes is positively correlated with the frequency of their ecDNA contacts (measured by the numbers of independent trans-interactions) (FIG. 9B). Taken together, ecDNA connectivity was highly associated with transcriptional activity, and a highly enhanced H3K27ac signature, suggesting that ecDNA can act as a global transcriptional amplification machinery.

In total across the four ecDNA (+) cell lines, 4,763 genes were in contact with ecDNA, of which 877 (18%) were shared by two or more cell lines (FIG. 3A). The function of these 877 genes was significantly enriched in translation initiation (FDR 2.06E-04), cell-cell adhesion (FDR 0.003) and transcription (FDR 0.02), biological processes involved in cell communication and proliferation (DAVID online analysis [Huang da, W. et al. (2009) Nat Protoc 4, 44-57]). Among the twenty genes commonly found in all four ecDNA (+) cell lines, over half are genes functionally associated with tumorigenesis, regulation of apoptosis, cell growth or proliferation, including ERBB2, DNAJB4, MCL1, DDIT4 and BAD, JUND and its transcription co-factor FOS, and non-coding RNA gene MALAT1. Examination was performed of the presence of a set of 736 chromosomal oncogenes [Forbes, S. A. et al. (2015) Nucleic Acids Res 43, D805-811; Repana, D. et al. (2019) Genome Biol 20, 1] in ecDNA-connectivity networks and it was found that respectively 87, 56, 78 and 54 annotated oncogenes were within the ecDNA-mediated chromatin interactomes across the four ecDNA (+) cell lines (FIG. 3B). This represented a 2.1- to 2.5-fold enrichment over random expectation (P<0.05; one-sided Wilcoxon rank-sum test), supporting the hypothesis that ecDNA recruits additional oncogenes through chromatin interactions for co-activation in cancer cells. Consistent with the effects of ecDNA on transcription activation, trans-interacting oncogenes showed six- to ten-fold FPKM increases compared to median transcription levels across all genes in each of the four ecDNA (+) lines (P values 1.3E-14 to 2.1E-24, one-sided Wilcoxon rank-sum test) (FIG. 9C). In total, it was determined that 216 of 736 annotated oncogenes exhibited trans-chromosomal interactions with ecDNAs in at least one of four ecDNA (+) cell lines (FIG. 3B). Among them, ERBB2 and MALAT1 exhibited chromatin connection with ecDNAs in all four neurospheres lines. ERBB2 is a canonical oncogene in breast cancer and shares structural similarities with EGFR, which is altered in 55%-60% of glioblastoma. ERBB2 and EGFR may form heterodimers to activate its downstream signaling pathway [Qian, X., et al. (1994) Proc Natl Acad Sci USA 91, 1500-1504]. ERBB2 is rarely genomically altered in GBM and is expressed in a nearly half of GBM [Zhang, C. et al. (2016) J Natl Cancer Inst 108, doi:10.1093/jnci/djv375; Liu, G. et al. (2004) Cancer Res 64, 4980-4986] but not in non-neoplastic brain cells (GEPIA online data [Tang, Z. et al. (2017) Nucleic Acids Res 45, W98-W102]). MALAT1 expression in GBM may result in WNT signaling [Vassallo, I. et al. (2016) Oncogene 35, 12-21] which drives endothelial trans-differentiation and increased migratory potential [Hu, B. et al. (2016) Cell 167, 1281-1295].

Beyond the recruitment of individual oncogenes, ecDNAs also appear to be the focal points where many oncogenes are brought together into spatial proximity via their interactions with ecDNAs. Among the 8, 11, 10 and 4 genome-wide interaction networks, defined by the extensive communication between hubs, in each of the four ecDNA (+) cell lines (Methods, FIG. 3C), all except 3 in HF-2927 harbor interaction hubs from ecDNAs. Many of these ecDNA-connected oncogenes reside within each of the chromatin networks. Specifically, individual communities in HF-3016 and HF-3177 can connect up to 10 and 12 additional oncogenes (FIG. 9D), which are significantly higher than random expectation (P<0.05; one-sided Wilcoxon rank-sum test). Such co-aggregation of oncogenes was indicated to be a structure-based mechanism adopted by ecDNAs to achieve coordinated transcriptional co-activation to promote tumorigenesis. Notably, despite the ecDNAs from HF-3016 and HF-3177 were derived from the primary and recurrent GBM of the same patient, the combinatorial coherence of ecDNA networks do not harbor many overlapping genes, implying that the heterogeneity of cancer clones can be further expanded by ecDNA-chromatin networks.

In summary, studies were performed using a chromatin interaction assay, ChIA-PET assay, to characterize the ecDNA transcription interactomes and regulation in cancer cells. Through the multi-omics integrative analysis of genome-wide ecDNA connectivity foci, trans-interacting chromosomal target genes, H3K27ac binding and RNA expression, it was demonstrated that ecDNAs can function as mobile enhancer elements that may preferentially target oncogenes for transcription co-activation in cancer cells (FIG. 9E). These findings outline a new ecDNA mechanism that provides cancer cells with a competitive advantage to drive tumor progression and tumor evolution. Furthermore, the identification of ecDNA targeted oncogenes can reveal candidates for targeted inhibition treatment strategies and the oncogene clusters transcriptionally activated by the ecDNA may be a mechanism to prioritize their effectiveness for a given tumor type.

In addition to offering the detailed characterization of ecDNA targeted chromatin interactomes in cancer genomes, the use of a chromatin interaction assay, such as but not limited to a ChIA-PET assay, provides an effective means to precisely map the amplified genomic domains within ecDNAs based on their intensive chromatin contacts with the linear chromosomes. Existing methods adopted to characterize ecDNA are either through imaging-based analysis [Turner, K. M. et al. (2017) Nature 543, 122-125] or structural analysis of regions with copy number gain [Deshpande, V. et al. (2018) bioRxiv doi.org/10.1101/457333]. Comparing with these methods, direct measuring the inter-chromosomal chromatin contact frequencies through chromatin interaction assays, non-limiting examples of which are ChIA-PET and Hi-C, offers an unbiased approach to uncover ecDNA signatures of different sizes, copy numbers or sequence context. Furthermore, the contact frequency and pattern between different regions of ecDNA molecules provides insight into their physical structure and continuity, much like the ability of 3D chromatin conformation to aid the characterization of genome structural variation and assembly [Spielmann, M. et al. (2018) Nat Rev Genet 19, 453-467; Dixon, J. R. et al. (2018) Nat Genet 50, 1388-1398].

Taken together, the experimental results demonstrated that ecDNAs can enhance expression of extrachromosomal and chromosomal gene transcription through chromatin interactions. Combined with the prevalence and diversity of ecDNA, these findings provide yet another level of complexity of ecDNA effects in cancer. Importantly, the results provide insights into the interplay between genetic structure and the epigenetic consequences in tumor evolution. Given the prevalence of ecDNA in cancers and the unique genomic dynamics of this extrachromosomal structure, this supports targeting ecDNA and their activated chromosomal genes in therapeutics.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

Where a range of values is provided, it is understood that each intervening value is encompassed. Where the stated range includes one or both of the limits, ranges excluding either or both of those included limits are also included in the invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of identifying an extrachromosomal DNA (ecDNA) in a cell, comprising:
(a) detecting a chromatin interaction between a non-linear DNA molecule and at least one linear chromosome of a chromosome pair, wherein the interaction comprises a contact between the non-linear DNA molecule and the at least one linear chromosome of the chromosome pair, and wherein the presence of:
(i) a significantly high frequency of the detected chromatin interaction in the cell;
(ii) contact between the non-linear DNA molecule and at least one linear chromosome of each of the chromosome pairs in the cell; and
(iii) an increase in an average per-cell number of copies of the non-linear DNA molecule in a plurality of the cells over time, identifies the non-linear DNA molecule as an ecDNA.

2. The method of claim 1, further comprising determining a frequency of the detected chromatin interaction.

3. The method of claim 1, further comprising determining size of the non-linear DNA.

4. The method of claim 1, further comprising determining a number of copies of the non-linear DNA molecule in the cell.

5. The method of claim 1, further comprising determining an average per-cell number of copies of the non-linear DNA molecule in a plurality of the cells at a first time and comparing the determined average to a control average per-cell number of copies of the non-linear DNA.

6. The method of claim 5, wherein the control average per-cell number of copies of the non-linear DNA molecule is an average per-cell number of copies of the non-linear DNA molecule determined in the plurality of cells at a different time point.

7. The method of claim 1, further comprising determining the sequence of at least a portion of the non-linear DNA.

8. The method of claim 7, further comprising identifying the presence of an oncogene sequence in the determined sequence.

9. The method of claim 1, wherein the cell is a cancer cell.

10. The method of claim 9, wherein the cell is obtained from a plurality of cells comprising the cancer cells.

11-15. (canceled)

16. A method of identifying an ecDNA-modulated oncogene, the method comprising:
(a) detecting an interaction between an ecDNA and one or more target genes located in a cell, wherein the detecting comprises directly measuring chromatin interactions between the ecDNA and a regulatory element of the one or more target genes;
(b) identifying one or more of the target genes of the interaction detected in step (a) in which the target gene's transcription is modulated by the detected interaction; and
(c) determining whether one or more of the target genes identified in step (b) is an oncogene, wherein the determination of the target gene as an oncogene identifies the oncogene as an ecDNA-modulated oncogene.

17. The method of claim 16, wherein the identifying in step (b) comprises measuring a level of transcription of the identified target gene and comparing the measured level to a control level of transcription of the target gene.

18-20. (canceled)

21. The method of claim 16, wherein the regulatory element comprises a promoter for the target gene.

22. The method of claim 16, wherein the target gene is located on a linear chromosome.

23. The method of claim 16, wherein the target gene is located on a second ecDNA.

24. The method of claim 16, wherein the cell is a cancer cell.

25-30. (canceled)

31. A method of determining an oncogene status of a cancer, the method comprising:
(a) identifying in a cancer cell an oncogene modulated by an ecDNA, and (b) determining one or more of a level and an effect of the ecDNA modulation of the oncogene as a determination of the oncogene status of the cancer.

32. The method of claim 31, wherein a means of identifying in step (a) comprises:
(i) detecting an interaction between an ecDNA and one or more target genes located in DNA of the cancer cell, wherein the detecting comprises directly measuring chromatin interactions between the ecDNA and a regulatory element of the one or more target genes;
(ii) identifying the target genes in the interactions detected in step (i) in which the target gene's or genes' transcription is modulated by the detected interaction; and
(iii) determining whether one or more of the target genes identified in step (ii) is an oncogene, wherein the determination of the target gene as an oncogene identifies the oncogene as an ecDNA-modulated oncogene in the cancer cell.

33. The method of claim 32, wherein the identifying in step (ii) comprises measuring a level of transcription of the identified target gene and comparing the measured level to a control level of transcription of the target gene.

34-48. (canceled)

49. The method of claim 31, wherein the cell is a cancer cell, the cell is obtained from a plurality of cells comprising the cancer cells, and the method further comprises: repeating steps (a) and (b) in a cancer cell obtained from a second plurality of cells comprising the cancer, and comparing one or more the level or effects detected in the cancer cell obtained from the first plurality of cells to the level or effects, respectively, detected in the cancer cell obtained from the second plurality of cells, wherein a difference in one or both of the level and effects indicates a change in the oncogene status of the cancer.

50-78. (canceled)
